(19) United States
(12) Patent Application Publication
(10) Pub. No.: US 2005/0221500 A1
(43) Pub. Date: Oct. 6, 2005

(54) Protein identification from protein product ion spectra

(75) Inventors: Gavin E. Reid, Victoria (AU); Jason M. Hogan, Lafayette, IN (US); Scott A. McLuckey, West Lafayette, IN (US)

Correspondence Address: MUETING, RAASCH & GEBHARDT, P.A., P.O. BOX 581415, MINNEAPOLIS, MN, 55458, US

(73) Assignee: Purdue Research Foundation, West Lafayette, IN (US)

(21) Appl. No.: 10/514,693

(22) PCT Filed: May 20, 2003
(86) PCT No.: PCT/US03/16029

§ 371 Date: Nov. 17, 2004

Related U.S. Application Data
(60) Provisional application No. 60/382,062, filed on May 20, 2002.

(51) Int. Cl. G06F 19/00; G01N 33/48; G01N 33/50

(52) U.S. Cl. 436/86; 702/19

(57) ABSTRACT

Mass spectrometry is used to identify a protein of interest. The protein is first ionized then fragmented into protein product ion. Masses of the observed product ions are compared to product ion masses calculated in silico for database protein sequences to identify product ion matches within a predetermined mass tolerance. An algorithm that weights the product ion to matches based upon one or more factors such as product ion abundance, favored cleavage sites, product ion type, precursor ion charge state and polarity is used to score the possible matches to database proteins in order to identify the protein of interest. The invention represents a “top down” approach and is particularly well-suited for identification of a protein in a complex mixture.

This application claims the benefit of U.S. Provisional Application Ser. No. 60/382,062, filed 20 May 2002, which is incorporated herein by reference in its entirety.

### STATEMENT OF GOVERNMENT RIGHTS

This invention was made with government support under a grant from the National Institutes of Health, Grant No. GM45372. The U.S. Government has certain rights in this invention.

### BACKGROUND OF THE INVENTION

Over the last fifteen years, mass spectrometry has played an increasingly important role in the identification of molecules of biological interest (Mann et al., Annu. Rev. Biochem. 2001, 70, 437-473). Indeed, recent developments in mass spectrometry have been the major factors enabling proteomics (Dove, Nature Biotechnol. 1999, 17, 233-236; Blackstock et al., Trends Biotechnol. 1999, 17, 121-127; Pandey et al., Nature 2000, 405, 837-846; Anderson et al., FEBS Letters 2000, 480, 25-31). In particular, the speed, specificity, and sensitivity of mass spectrometry make it especially attractive for use in strategies requiring rapid protein identification and characterization.

Protein identification by database searching can be either peptide or protein based. To date, the most extensively employed methodologies for complex protein mixture analysis have been initiated by one- or two-dimensional gel electrophoresis, followed by proteolytic digestion of individual protein spots or gel slices. Protein identification is then accomplished by peptide mass fingerprinting, in the case of pure proteins or simple protein mixtures, or by tandem mass spectrometry (MS/MS) of individual peptides followed by protein sequence database analysis of the product ion spectra, in the case of those proteins present in more complex mixtures. Peptide-based methods for protein identification using mass spectrometry are summarized below.

Protein Identification Based on Mass Spectrometry Information Obtained from Proteolytically Derived Peptide Ions

The peptide-based, or “bottom up”, approach to protein characterization involves digesting the protein into peptide fragments prior to database searching. Conventional approaches to protein identification by database searching generally involve using data obtained by mass spectrometry of chemically or proteolytically derived peptides (Aebersold et al. Chem. Rev. 2001, 101, 269-295). The proteolytic enzyme trypsin is most commonly used for this purpose as it specifically cleaves at the relatively common amino acid residues lysine and arginine to produce peptides with good ionization characteristics as well as being amenable to subsequent dissociation to yield structural information. Several variations can be used to link mass spectral data with entries in protein sequence databases.

A major advantage of this overall approach is that it does not require extensive sequence information. In fact, some strategies do not even require the direct extraction of any sequence information from the mass spectral data. The combination of mass spectral data with protein database information provides a rapid means for identifying the gene from which a gene product is derived and, in favorable cases, some information regarding the actual identity of the gene product. However, information present in databases is, to varying degrees, incomplete and inaccurate in relation to the mature expressed protein, due to the multitude of post-translational processing events that can occur after protein translation. Nevertheless, the combination of mass spectral data with protein database information can be used to improve the quality of information in the databases and currently constitutes the most efficient approach, with respect to both time and sample consumption, for the identification of proteins in complex mixtures.

Protein Identification Based on Mass Fingerprinting of Chemically or Proteolytically Derived Peptide Ion Masses

One strategy for protein identification using mass spectrometry derived data is termed “peptide mass fingerprinting” (Henzel et. al. Proc. Natl. Acad. Sci. USA. 1993, 90, 5011-5015. James et. al. Biochem. Biophys. Res. Commun. 1993, 195, 58-64; Mann et al. Biol. Mass Spectrom. 1993, 22,338-345; Pappin et al. J. Curr. Biol. 1993, 3, 327-332; Yates et al. Anal. Biochem. 1993, 214, 397-408; Zhang et al. Anal. Chem. 2000, 72, 2482-2489; Clauser et al. Anal. Chem. 1999, 71, 2871-2882). Following digestion of the protein, the molecular masses of the peptides are determined. These masses are characteristic of the protein, and provide a peptide “mass fingerprint” which can be used in database searches to subsequently identify the protein. Prior to database searching, the protein sequences in the database, or a subset of these proteins extracted from the database based on constraints such as the experimentally observed mass range or isoelectric point of the intact protein, are digested in silico according to the specificity of the enzyme used, into their corresponding peptides. The experimentally determined peptide masses are then compared to the masses of these theoretical peptides. Proteins are ranked based on the number of peptides from a given protein in the database that match to the experimental peptide masses.

Note that protein digestion rarely provides 100% sequence coverage of the protein, due to losses of some peptides during sample handling prior to mass spectrometry, or due to the masses of the peptides falling outside the observable mass range of the instrument, therefore making complete protein characterization difficult to achieve. Additionally, while the approach is also amenable to the analysis of simple protein mixtures, provided that sufficient peptide masses are obtained to unambiguously identify each component of the mixture, peptide mass fingerprinting is generally not suited to the analysis of peptides resulting from proteolysis of complex protein mixtures, as the presence of peptides from many different proteins makes it difficult to assign individual peptides to their correct proteins.

Protein Identification Based on Derivation of a Sequence Tag from Peptide Product Ion Spectra

A more comprehensive approach to protein identification, particularly for individual proteins present in complex mixtures, is to subject each of the proteolytically derived peptides to tandem mass spectrometry and to derive the protein identity based on interpretation of the resultant product ion spectra. While it is generally difficult to derive a complete peptide sequence from an MS/MS spectrum, it is often straightforward to derive 3 or 4 residues of contiguous amino acid sequence data from a series of product ions corresponding to fragmentations at adjacent residues, thereby providing a “sequence tag” suitable for database interrogation (Mann et al. Anal. Chem. 1994, 66, 4390-4399). In an approach initially described by Mann and Wilm, the sequence tag is combined with the masses of the “flanking” regions i.e., the N- and C-terminal masses on either side of the sequence tag, as well any supplemental information such as the specificity of the enzyme used to generate the peptide, then compared against theoretical peptides generated from the database. Importantly, this method is error tolerant as one, or several, of the regions of the sequence tag (the tag itself or the flanking mass regions) may contain errors due to post-translational modifications yet still result in an unambiguous assignment. The sequence tag process has been recently been further refined by Pappin and co-workers (Pappin et al. Mass Spectrom. Biol. Sci., 1996, 135-150) to include the use of product ion intensity values to select sub-sets of the most intense peaks outside of the assigned sequence tag region to improve scoring discrimination. However, as the sequence tag approach requires some user intervention prior to database analysis, it has not generally been employed for large scale, high throughput applications.

Protein Identification by Database Interrogation of Uninterpreted Product Ion Spectra.

Perhaps the most widely employed approach for database interrogation using peptide ion MS/MS data has been to directly subject the data to interrogation without any prior user interpretation (Eng et al. J. Am. Soc. Mass Spectrom. 1994, 5, 976-989; Yates et al. Anal. Chem. 1995, 67, 3202-3210; Yates et al. Anal. Chem. 1995, 67, 1426-1436; PROSPECTOR, available on the worldwide web at prospector.ucsf.edu; PROFOUND, available on the worldwide web at 65.219.84.5/Proteinld.html or prowl.Rockefeller.edu; MASCOT, available on the www.matrixscience.com). This approach involves extracting from the database those peptides matching the experimentally observed mass, using constraints such as the specificity of the enzyme used, and then comparing the observed product ion masses against theoretical spectra generated from the extracted peptide sequences. A range of search constraints, as well as incorporation of known or predicted post-translational modifications can be used to increase the search specificity. The SEQUEST program developed by Eng and Yates (Eng et al. J. Am. Soc. Mass Spectrom. 1994, 5, 976-989; Yates et al. Anal. Chem. 1995, 67, 3202-3210; Yates et al. Anal. Chem. 1995, 67, 1426-1436) is the most widely employed version of this approach. SEQUEST selects the 200 most abundant peaks from an experimental peptide MS/MS spectrum and normalizes the abundances to 100. Protein sequences from a database are scanned for sequences of amino acids that match the experimental peptide mass within a tolerance range. The scoring algorithm proceeds by summing the normalized abundance values for all experimental fragment ions that match predicted fragment ions. Weighting factors for consecutive fragment ions, and immonium ions for histidine, tyrosine, tryptophan, methionine, and phenylalanine when observed with the amino acid fragment are included. Negative weighting occurs when the immonium ion is observed without the amino acid. The final total of normalized abundances and weighting factors are divided by the total possible fragment ions to determine a score. A cross-correlation score is determined using the top 500 identified amino acid sequences from the experimental data. The cross-correlation score describes how well the virtual spectrum for each sequence matches the observed spectrum.

Protein Identification by “De Novo” Sequence Analysis of Peptide Product Ion Spectra

This approach involves the derivation of the complete primary sequence of a peptide from the product ions formed via MS/MS and MSⁿ (Hunt et al. Proc. Natl. Acad. Sci. USA. 1986, 83, 6233-6237; Biemann et al. Acc. Chem. Res. 1994, 27, 370-378; Papayannopoulos Mass Spectrom. Rev. 1995, 14, 49-73; Cox et al. Science. 1994, 264, 716-719; Hunt et al. Science. 1992, 255, 1261-1266; Wang et al. Science. 1995, 269, 1588-1590). The advantage of this approach is that it provides complete primary structure information for the peptide, including the identity and location of any post-translational modifications. However, this approach often requires additional information supplied by, for example, a single cycle of Edman degradation to determine the order of the two N-terminal amino acids, or derivatization via acetylation or methylation to distinguish amino acids such as glutamine and lysine, or aspartic acid and glutamic acid from asparagine and glutamine, respectively. Hence, the approach is generally considered to be more labor intensive and expensive in terms of both time and sample consumption than the database searching approaches described above.

More recently, significant effort has been expended toward the development of automated software for de novo interpretation of peptide product ion spectra (Taylor et al. Rapid Comm. Mass Spec. 1997, 11, 1067-1075; Taylor et al. Anal. Chem. 2001, 73, 2594-2604; Dancik et al. J. Comp. Biol. 1999, 6, 327-342; Chen et al. J. Comp. Biol. 2001, 8, 325-337). However, the utility of de novo peptide sequence interpretation methods is heavily dependent on the peptide ion yielding sufficient product ions to allow derivation of its complete sequence. Several studies have indicated that a significant proportion of peptides subjected to dissociation yield insufficient product ions, product ions corresponding to cleavages not included in the search algorithms, or lack product ions with sufficient signal-to-noise, to allow their identification (Simpson et al. Electrophoresis 2000, 21, 1707-1732).

Unfortunately, several classes of proteins, notably hydrophobic proteins, low abundance proteins, and those with extremes of isoelectric point (pI) and molecular weight are poorly represented in 2D-gel based separations (Gygi et al., Proc. Natl. Acad. Sci. U.S.A. 2000, 97, 9390-9395). A number of multidimensional chromatographic approaches for extensive separation of the peptides resulting from digestion of unfractionated complex protein mixtures have been described, resulting in a substantial increase in the number of proteins that can be identified during the course of a single analysis, and also overcoming many of the protein discrimination effects associated with gel based protein identification strategies (Davis et al., J. Chromatogr., B: Biomed. Sci. Appl. 2001, 752, 281-291; Washburn et al., Nat. Biotechnol. 2001, 19, 242-247; Wolters et al., Anal. Chem. 2001, 73, 5683-5690). However, digestion of an unfractionated protein mixture greatly increases the number of components to be analyzed and condenses the resultant peptide mixture into a narrow mass range, thereby complicating the task of isolating individual components for further analysis, placing greater demands on the performance of the mass spectrometer. Furthermore, a problem common to all peptide sequencing approaches is that MS/MS spectra often yield insufficient product ions, product ions corresponding to cleavages not included in the search algorithms, or lack product ions with sufficient signal-to-noise, to allow their identification (Simpson et al., Electrophoresis 2000, 21, 1707-1732). Finally, it is common that many of the peptides resulting from digestion are not observed, making complete characterization of the protein difficult to achieve.

An alternate approach for the rapid identification and characterization of proteins, termed “top down” protein characterization (Kelleher et al. J. Am. Chem. Soc. 1999, 121, 806-812), involves the fragmentation of whole protein ions in the gas-phase without prior recourse to enzymatic digestion or extensive separation steps. Provided that sufficient fragmentation occurs, the protein may be identified by the “sequence tag” strategy (Mortz et al., Proc. Natl. Acad. Sci. U.S.A. 1996, 93, 8264-8267; Cargile et al., Anal. Chem. 2001, 73, 1277-1285; Demirev et al., Anal. Chem. 2001, 73, 5725-5731), via database searching of the uninterpreted product ion spectrum (Meng et al., Nat. Biotechnol. 2001, 19, 952-957), or through determination of the complete amino acid sequence (Horn et al., Proc. Natl. Acad. Sci. USA 2000, 97, 10313-10317; Horn et al., J. Am. Soc. Mass Spectrom. 2000, 11, 320-332.).

A major potential advantage of this strategy over the peptide based “bottom up” approach is that performing an MS/MS experiment on an intact protein ion makes the entire sequence available for examination, allowing, for example, the facile identification and characterization of post-translational modifications (Meng et al., Nat. Biotechnol. 2001, 19, 952-957; Reid et al., Anal. Chem. 2001, 74, 577-583; Kelleher et al., Anal. Chem. 1999, 71, 4250-4253; Fridriksson et al., Biochemistry. 2000, 39, 3369-3376; Shi et al., Anal. Chem. 2001, 73, 19-22). Furthermore, a top-down approach to protein mixtures can circumvent complications associated with proteolytic digestion, such as the creation of a complex peptide mixture and the compression of all mixture components into a relatively narrow mass window. Additionally, the many redundant protein identifications that are often associated with MS/MS of proteolytically derived peptides can be avoided. Current “top down” approaches are summarized below.

Protein Identification Based on Database Analysis of Information Obtained from the Dissociation of Whole Protein Ions

While protein mass is fundamentally informative, it alone is not particularly useful in unambiguously identifying a protein due to the potential for post-translational processing to cause differences between the predicted and experimentally observed masses. However, partial protein sequence information in combination with intact protein mass can provide a level of insight not available from either piece of information alone, and can potentially provide significantly more information than that provided from the dissociation of proteolytically derived peptides. “Top down” approaches for protein identification and characterization (Kelleher et al. J. Am. Chem. Soc. 1999, 121, 806-812) have recently been developed that enable primary structural information to be obtained directly from the gas-phase dissociation of intact protein ions without prior recourse to extensive separation or digestion. Using this approach, ions derived from the intact protein are fragmented in the mass spectrometer, and if necessary, the resulting product ions are subjected to further fragmentation until sufficient product ions are generated to allow for the identification of the protein.

Protein Identification Based on “De Novo” Sequence Analysis of Whole Protein Ion Dissociation Spectra

Similar to that described above for de novo interpretation of peptides based on the interpretation of the product ion spectrum, de novo approaches have also been applied to the analysis of whole protein ion MS/MS product ion spectra. However, this approach to date has required the use of multiple activation methods and has been completely successful for only a very small number of proteins. The de novo sequencing approach for proteins is facilitated by the complementary nature of the product ions formed by collision-activated dissociation (CAD) (b- and y-type ions) versus electron capture dissociation (ECD) (predominantly c and z type ions) in Fourier transform ion cyclotron resonance (FT-ICR) instruments that allows the identities of these ions to be readily assigned. Software for automated de novo interpretation of the complex product ion spectra arising from CAD and ECD data has now been developed. For example, de novo assignments of the complete sequences of ubiquitin (8.6 kDa), mellitin (2.8 kDa), and 91% of the sequence of cytochrome c (12.4 kDa) have recently been demonstrated using this approach (Horn et al. Proc. Natl. Acad. Sci. USA 2000, 97, 10313-10317).

Protein Identification Based on Derivation of a Sequence Tag from the Dissociation of Whole Protein Ions

In analogy with the sequence tag approach described above for linking peptide derived mass spectral data to protein database information, a similar approach has been utilized for protein identification based on derivation of a sequence tag from the product ion spectrum of whole protein ions (Mortz et al. Proc. Natl. Acad. Sci. U.S.A. 1996, 93, 8264-8267). It has previously been demonstrated that database analysis of a sequence tag derived from the product ions formed by dissociation of the intact multiply charged precursor ions of bacteriophage MS2 viral coat protein expressed in E. coli (Cargile et al. Anal. Chem. 2001, 73, 1277-1285) could identify the protein. In this work, a small number of precursor ion charge states (+7 to +9) were examined and a number of sequence tags from each charge state used to search the entire SwissProt database. In conjunction with the measured intact protein mass, the sequence tag searches resulted in the retrieval of two proteins from the database, differing by only one amino acid (an aspartic acid to asparagine substitution).

Fenselau and coworkers have recently used sustained off-resonance irradiation/CAD and ECD in a Fourier transform ion cyclotron resonance (FT-ICR) mass spectrometer, coupled with the derivation of a sequence tag to identify the major protein biomarker from an extract of Bacillus cereus T-spores. The sequence tag was used in a BLAST search of the entire SwissProt and TrEMBL databases to unambiguously identify the ˜7 kDa protein, as well as its methionine oxidized derivative (Demirev et al. Anal. Chem. 2001, 73, 5725-5731). In a recent study employing FF-ICR MS in conjunction with CAD and ECD to characterize large proteins (45 kDa), McLafferty and coworkers have employed the sequence tag approach to identify a Bacillus subtilis protein overexpressed in E. coli prior to characterization of its post translational modifications. In this work, a C-terminal sequence tag that uniquely matched one of the enzymes for thiamine biosynthesis, ThiS, from the translated genomic database was used to uniquely identify this protein (Ge et al. J. Am. Chem. Soc. 2002, 124, 672-678).

Note that the sequence tag method relies on fragmentation occurring along a contiguous stretch of the protein ion, a condition not always met when subjecting protein ions to dissociation. A number of recent studies have indicated that the fragmentation of whole protein ions is strongly influenced by the precursor ion charge state, as well as the total number of basic sites in the amino acid sequence (Cargile, Jr. et al. Anal. Chem. 2001, 73, 1277-1285, 34-39). Generally, intermediate charge states give rise to the most extensive non-specific cleavage of the protein backbone, often allowing derivation of a sequence tag for subsequent database searching. At other charge states, the facile loss of NH₃ or H₂O (very low charge states corresponding to less than the number of arginine residues), preferential cleavage at the C-terminal of aspartic acid and lysine residues (low charge states), and preferential cleavage at the N-terminal of proline residues (high charge states), are often the dominant fragmentation products observed (Reid et al. Anal. Chem. 2001, 73, 3274-3281; Engel et al., Int. J. Mass Spectrom. 2002, 219, 171-187; Newton et al., Int. J. Mass Spectrom. 2001, 212, 359-376).

Protein Identification by Database Interrogation of Uninterpreted Whole Protein Product Ion Data

The uninterpreted product ion database search approach described above for the identification of peptide product ion spectra has also been used to match the observed product ions from whole protein ion dissociations. Early work by Marshall and coworkers (Li et al., Anal. Chem. 1999, 71, 4397-4402) involving the dissociation of a number of standard proteins discussed an approach whereby a list of candidate proteins within a specified mass range about the accurately measured intact protein mass were initially retrieved from the database. The masses of the b- and y-type product ions were then matched to the predicted product ion mass values of each of the retrieved proteins. A scoring scheme based on the number of “hits” as well as a “sequence tag” score was shown to correctly identify these proteins from over 1000 protein database entries that were within ±500 Da of the experimentally observed intact protein mass.

Using a similar approach, Kelleher and coworkers have recently demonstrated the identification of a number of a priori unknown proteins using only three to four nonadjacent fragment ions with no intact protein mass constraints, by FT-ICR MS. They also developed a scoring algorithm that assigned each product ion corresponding to cleavage along the protein backbone a point value, with a larger point value assigned for product ions corresponding to cleavage at preferred fragmentation sites (N-terminal to proline, and C-terminal to aspartic and glutamic acid) (Meng et al., Nat. Biotechnol. 2001, 19, 952-957). Fragmentations corresponding to preferred cleavage sites (N-terminal to proline, C-terminal to aspartic acid and glutamic acid) received 7 points while any other cleavage received 2ⁿ points (where n is equal to the number of matched fragments), with a bonus score of 2^{j+1} (where j is equal to the number of matched fragments), weighted for fragmentations occurring within 3 amide bonds of a preferred cleavage.

A major issue associated with implementation of the top-down approach on most types of tandem mass spectrometers is that the spectra derived from the dissociation of multiply-charged proteins ions typically contain product ions with charge states ranging from +1 up to the charge of the precursor ion, thereby creating possible ambiguities in assigning product ion mass and charge. High magnetic field strength Fourier transform ion cyclotron resonance (FIT-ICR) mass spectrometry (Marshall et al., Mass Spectrom. Rev. 1998,17,1-35), which enjoys sufficient resolving power (typically >10⁵) to enable the measurement of the isotope spacings of high mass product ions, allows for the interpretation of product ion spectra such that reliable structural information on intact proteins as large as 45 kDa can be obtained (Ge et al., Am. Chem. Soc. 2002, 124, 672-678; Ge et al., Int. J. Mass Spectrom. 2001, 2101211, 203-214; Forbes et al., Proteomics. 2001, 1, 927-933; Fridriksson et al., Biochemistry 2000, 39, 3369-3376; Horn et al., Anal. Chem. 2000, 72, 4778-4784; Kelleher et al., Protein Sci. 1998, 7, 1796-1801).

Two recent papers described above have highlighted the utility of the FT-ICR MS approach for the identification of unknown proteins from simple mixtures. Kelleher and coworkers (Meng et al., Nat. Biotechnol. 2001, 19, 952-957) demonstrated the identification of 18 archaeal and bacterial proteins ranging from 7 to 36 kDa present in a mixture of modest complexity, where only three or four non-adjacent fragment ions with a mass accuracy of ±0.1 Da were required for a 99% confidence of a correct match from a database containing 5,000 proteins, with no intact mass bias required for protein identification. Fenselau and coworkers used intact protein ion dissociation and database searches to identify the major biomarker derived from an extract of Bacillus cereus T-spores (Demirev et al., Anal. Chem. 2001, 73, 5725-5731). In this example, a sequence tag was used to conduct BLAST searches of the entire SWISSProt/TrEMBL database to unequivocally identify the protein of interest (about 7 kDa), as well as its methionine oxidized derivative.

A gas-phase chemical approach to address the issue of product ion charge state determination has been implemented in a quadrupole ion trap mass spectrometer, whereby the multiply charged product ions are reduced to their singly charged forms by ion/ion proton transfer reactions with singly charged ions of the opposite polarity (Stephenson et al., Anal. Chem. 1998, 70, 3533-3544). Conversion of product ions to the +1 charge state after parent ion dissociation greatly simplifies the interpretation of protein ion MS/MS spectra. Interpretable MS/MS spectra of proteins of up to 20 kDa, for example, have been obtained using ion trap instruments with limited mass resolution (e.g., M/ΔM=0.5-2×10³) (Cargile et al., Anal. Chem. 2001, 74, 577-583; Stephenson et al., Anal. Chem. 1998, 70, 3533-3544; Stephenson et al., Rapid Commun. Mass Spectrom. 1999, 13, 2040-2048; Schaaff et al., Anal. Chem. 2000, 72, 899-907; Wells et al., Int. J. Mass Spectrom. 2000, 203, A1-A9; Reid et al., Anal. Chem. 2001, 73, 3274-3281; Wells et al., J. Am. Soc. Mass Spectrom. 2001, 12, 873-876; Engel et al., Int. J. Mass Spectrom. 2001, In Press; Chrisman et al., Rapid Commun. Mass Spectrom. 2001, 15, 2334-2340; Newton et al., Int. J. Mass Spectrom. 2001, 212, 359-376). Ion/ion reactions may also be employed to form lower charge state precursor ions than those formed directly via electrospray ionization (Stephenson et al., J. Am. Chem. Soc. 1996, 118, 7390-7397; Stephenson et al., Anal. Chem. 1996, 68, 4026-4032), thereby allowing access to the additional structural information imparted by dissociation of these charge states (Wells et al., Int. J. Mass Spectrom. 2000, 203, A1-A9; Reid et al., Anal. Chem. 2001, 73, 3274-3281; Engel et al., Int. J. Mass Spectrom. 2001, In Press; Newton et al., Int. J. Mass Spectrom. 2001, 212, 359-376). A number of recent studies on a wide range of precursor ion charge states from standard proteins such as mellitin (Stephenson et al, Anal. Chem. 1998, 70, 3533-3544), lysozyme (Stephenson et al., Rapid Commun. Mass Spectrom. 1999, 13, 2040-2048), hemoglobin β-chain (Schaaff et al., Anal. Chem. 2000, 72, 899-907), insulin (Wells et al., Int. J. Mass Spectrom. 2000, 203, A1-A9), ubiquitin (Stephenson et al., Anal. Chem. 1998, 70, 3533-3544; Reid et al., Anal. Chem. 2001, 73, 3274-3281), cytochrome c (Wells et al., J. Am. Soc. Mass Spectrom. 2001, 12, 873-876; Engel et al., Int. J. Mass Spectrom. 2001, In Press), myoglobin (Chrisman et al., Rapid Commun. Mass Spectrom. 2001, 15, 2334-2340; Newton et al., Int. J. Mass Spectrom. 2001, 212, 359-376), and bacteriophage MS2 coat protein (Cargile et al., Anal. Chem. 2001, 73, 1277-1285) have demonstrated cleavage of greater than 50% of the protein backbone amide bonds from individual precursor ion charge states (Newton et al., Int. J. Mass Spectrom. 2001, 212, 359-376), with as high as 80% cleavage obtained from the information derived from several charge states (Engel et al., Int. J. Mass Spectrom. 2001, In Press). The sequence coverage can be further extended by the use of multistage MS/MS (MSn) of selected first generation product ions (Reid et al., Anal. Chem. 2001, 73, 3274-3281).

As the protein mixture complexity grows, it becomes increasingly likely that ions corresponding to proteins of different mass and charge will have the same nominal m/z values. Product ion spectra derived from an isolated precursor ion population could therefore include contributions from multiple proteins, thereby complicating protein identification. Ion/ion proton transfer reactions may also be used to overcome this potential complication, via manipulation of the precursor ion charges state prior to their dissociation (Cargile et al., Anal. Chem. 2001, 73, 1277-1285; Stephenson et al., J. Am. Soc. Mass Spectrom. 1998, 9, 585-596). In particular, ion/ion reactions can be used in a “double isolation” experiment, whereby an initial precursor ion selection step is followed by a short ion-ion reaction period and a second ion isolation step. If the second ion isolation window is chosen to correspond to the expected m/z change associated with proton transfer reactions of the protein of interest, all other proteins of different charge in the initially isolated m/z window will be resolved and ejected by the second isolation step, resulting in a “charge state purified” precursor ion population. This approach was first employed for identification of the bacteriophage MS2 virus that had been over expressed in E. coli (Cargile et al., Anal. Chem. 2001, 73, 1277-1285).

A limitation of the “double isolation” approach is that the ions from a given protein charge state are distributed over several charge states during the ion/ion reaction, thereby diluting the protein ion signal and decreasing the sensitivity for subsequent isolation and dissociation. Recently, however, it has been demonstrated that the rates of ion/ion reactions in a quadrupole ion trap may be selectively inhibited in a mass-to-charge selective fashion by the application of a resonance excitation voltage, tuned to the fundamental secular frequency of motion of an ion of interest, during the ion/ion reaction period. The inhibition of ion/ion reactions for selected ions, termed “ion parking,” (Shi et al., Anal. Chem. 2001, 73, 19-22) enables several analytically useful capabilities for the analysis of complex mixtures. In particular, inhibition of the ion/ion reaction rates at a specific m/z allows essentially all the ion current of a protein of interest to be concentrated into a single charge state, thereby overcoming the limitation outlined above.

Although techniques that increase “charge state purification” such as double isolation of the precursor ion and ion parking have made the “top down” approach to mass spectrometry-driven protein sequencing more reliable, difficulties in matching product ion spectra with protein database information still frequently arise, especially when the information in databases is incomplete and inaccurate in relation to the mature expressed protein. The identification and characterization of proteins present in complex mixtures remains an important analytical problem.

### SUMMARY OF THE INVENTION

The present invention provides a novel method for utilizing mass spectrometry to identify a protein of interest. The protein to be identified may be present in a mixture of proteins, or it may be isolated. The method of the invention is particularly well suited to identifying proteins in mixtures, including complex mixtures, that contain a multiplicity of proteins.

The protein of interest is subjected to tandem mass spectrometry such that it is ionized to form a protein precursor ion, then fragmented or dissociated into a multiplicity of protein product ions having experimentally determined product ion masses. When the method is performed on a mixture that includes a multiplicity of proteins, the population of protein precursor ions produced during the initial ionization of the complex mixture is preferably mass selected prior to dissociation/fragmentation into product ions.

Product ion masses can be experimentally determined from a product ion mass spectrum. Experimentally determined product ion masses are compared with product ion masses calculated for each member of a comparison set of database protein sequences, thereby elucidating, for each member of the comparison set, product ion matches that are within a predetermined mass tolerance. The number of product ion matches can be counted for each member of the comparison set, and matches or possible matches between the protein of interest and one or more members of the comparison set are thereby identified.

The comparison set of database proteins sequences may include all or only some of the protein sequences or subsequences included in one or more protein databases. For example, the comparison set may be limited to protein sequences or subsequences having a calculated mass that matches the mass of the protein of interest within a predetermined mass tolerance.

Optionally, product ion masses calculated for database protein sequences include product ion masses calculated for database protein sequences that have been modified to account for one or more known or predicted protein structural modification. The invention also includes modifying the database protein sequences to thereby expand the database to include such structurally modified proteins.

The comparison between experimentally observed product ion masses and calculated product ion masses can be made on the basis of either absolute mass or relative mass. The use of relational masses is especially suited to situations wherein the protein of interest is not or may not be accurately reflected in the protein database. Thus, in one embodiment of the invention, the analysis is made by first determining mass differences between selected pairs of experimentally determined product ion masses, and the mass differences between selected pairs of calculated product ion masses. The mass differences between the selected pairs of experimentally determined product ion masses and the mass differences between selected pairs of calculated product ion masses are then compared to identify for each member of the comparison set the product ion matches that fall within a predetermined mass tolerance. Pairs of product ions selected for the determination of mass differences can be selected on the basis of favored cleavage sites.

In a preferred embodiment, the method of the invention further includes discriminating between possible matches to members of the comparison set on the basis of experimentally observed product ion abundances, so as to identify the protein of interest. A score that includes a weighted sum of the product ion mass matches based on experimentally observed product ion abundances is calculated for each member of the comparison set.

Alternatively or in addition, the score can include a weighted sum of the product ion mass matches based on favored cleavage sites. The weighting factors assigned to the favored cleavage sites can vary with the identity of the amide bond. They can also be affected by the charge state of the multiply charged protein precursor ion formed in the initial ionization of the protein prior to fragmentation, and/or by the charge polarity (positive or negative) of the protein precursor ion. Charge polarity can also affect the type of product ion produced, and weighting factors can optionally be assigned to ion type as well.

When the protein of interest contains a disulfide linkage, a negatively charged protein precursor ion is typically produced during the initial ionization, and the resultant product ions typically include at least one z-S ion. The invention therefore further includes a method for identifying a protein containing a disulfide bond which includes subjecting the protein to tandem mass spectrometry to cause it to fragment into a multiplicity of product ions (including at least one z-S product ion) having experimentally determined product ion masses, and comparing the experimentally determined product ion masses with product ion masses calculated for each member of a comparison set that includes database protein sequences, so as to identify for each member of the comparison set the product ion matches within a predetermined mass tolerance. As noted above, a score can be calculated for each member of the comparison set which includes a weighted sum of the product ion mass matches based one or more factors such as experimentally observed product ion abundances, favored cleavages sites, precursor ion charge state and polarity, ion type, and the like.

As it has also been discovered by the inventors that the prevalence of favored cleavage sites can be affected by the charge state of the protein precursor ion, the invention also encompasses a method for identifying a protein of interest that assigns weighting factors to favored cleavage sites which vary with the charge state of the protein precursor ion. Thus, another embodiment of the method of the invention includes subjecting the protein of interest to tandem mass spectrometry to cause it to fragment into a multiplicity of product ions having experimentally determined product ion masses, wherein the protein of interest is ionized to yield a multiply charged protein precursor ion prior to fragmentation; comparing the experimentally determined product ion masses with product ion masses calculated for each member of a comparison set that includes database protein sequences, so as to identify for each member of the comparison set the product ion matches within a predetermined mass tolerance; and calculating a score for each member of the comparison set, wherein the score includes a weighted sum of the product ion mass matches based on favored cleavage sites, and wherein the weighting assigned to said favored cleavage sites depends upon the charge state of the protein precursor ion.

### BRIEF DESCRIPTION OF THE DRAWINGS

FIG. 1A is a schematic representation of the ion trap scan function used for gas-phase concentration, purification and dissociation of whole protein ions from complex mixtures.

FIG. 1B is a schematic representation of one method for generating a product ion spectrum: proteins in a complex mixture are ionized, and the resultant protein precursor ions are concentrated and/or purified using ion parking; subsequent dissociation of the precursor ion(s) allows protein identification based on the resultant product ion mass spectrum.

FIG. 2 shows fractionation of the soluble protein containing fraction from a whole cell lysate of E. coli by reverse phase high pressure liquid chromatography (RP-HPLC).

FIG. 3A shows pre-ion/ion reaction mass spectra of a fraction (retention time 9.0-9.5 minutes) from RP-HPLC of the E. coli whole cell lysate soluble protein fraction in FIG. 2.

FIG. 3B shows post-ion/ion reaction mass spectra of the same fraction.

FIG. 4A shows, for selected ions from the E. coli fraction shown in FIG. 3A, the mass spectrum obtained after a short ion/ion reaction period of isolated m/z region 1049 (±10 Da).

FIG. 4B shows ion parking of m/z 1468 during an ion/ion reaction on the isolated m/z region 1049.

FIG. 5A shows the post-ion/ion reaction CID MS/MS spectra of the [M+5H]⁵⁺ ion (m/z 1468 in FIG. 4B) of the protein at mass 7332 in FIG. 3B.

FIG. 5B shows the post-ion/ion reaction CID MS/MS spectra the [M+5H]⁵⁺ ion of the protein at mass 7273 in FIG. 3B.

FIG. 6A shows the post-ion/ion reaction CID MS/MS spectra of the [M+7H]⁷⁺ ion of the protein at mass 9740 in FIG. 3B.

FIG. 6B shows the post-ion/ion reaction CID MS/MS spectra of the [M+6H]⁶⁺ ion of the protein at mass 9065 in FIG. 3B.

FIG. 6C shows the post-ion/ion reaction CID MS/MS spectrum of the [M+5H]⁵⁺ ion of the protein at mass 6318 in FIG. 3B.

### DETAILED DESCRIPTION OF THE INVENTION

The present invention is directed to methods for identifying proteins, particularly those present in a complex mixture, using protein product ion data (also referred to herein fragment ion data) generated through mass spectrometry. Protein identification is based on the masses and, in a preferred embodiment, abundances of the product ions after fragmentation of the relatively large protein precursor (parent) ion, typically representing a whole protein or a large protein fragment. The database against which the product ion masses are matched is preferably annotated or adjusted to reflect known or expected structural variants of the database proteins, and may also include subsequences that represent fragments of database proteins.

The method of the invention is considered a “top down” approach and is particularly suited to use in proteomics applications. In the present method, proteins in a complex mixture do not need to be digested into small units (i.e., peptides) prior to the application of mass spectrometry; whole proteins can be analyzed directly. If it is necessary to reduce the size of the protein prior to ionization (due to instrumental constraints, for example), protein digestion techniques that lead to large protein fragments, such as digestion with cyanogen bromide, can be used since the large fragments can be directly analyzed without further cleavage.

It should be nonetheless understood, however, that there is no lower mass limit on the proteins, protein fragments or peptides that can be analyzed using the method of the invention. Likewise on the high mass end, the size of the protein or protein fragment is not limited except by the dynamic range of the mass spectrometer.

Any mixture containing a multiplicity of proteins can be analyzed in accordance with the method of the invention. Samples suitable for analysis include, without limitation, body fluids such as blood, serum and urine; tissue samples; and cell lysates, which can be, for example, bacterial cell lysates or eukaryotic cell lysates, particularly mammalian cell lysates such as from humans. Proteins in the sample need not be pre-treated to remove post-translational modifications or other structural modifications, although pre-treatment is of course not precluded by the invention.

Importantly, this novel approach to protein identification remains robust in the face of discrepancies between the database structure and the actual structure of the protein to be identified, including, for example, discrepancies resulting from co- or post-translational modifications, targeting sequences, pre- or prosequences; or variants due to differential processing of the mRNA or the mature protein.

Tandem mass spectrometry is preferred for use in the protein identification method of the invention, although the invention is not intended to be limited by the process used to produce the product ion spectrum. The important point is that the population of precursor ions produced during the initial ionization of the complex mixture is preferably mass selected prior to dissociation/fragmentation. Mass selection within a specified tolerance after ionization results in a precursor ion population that ideally contains one dominant precursor (parent) ion.

It should be noted that as complexity of the sample increases (for example, if the sample is a whole cell lysate), the chances that a precursor ion characterized by a given m/z within a specific tolerance actually represents multiple proteins also increases. Accordingly, the use of methods to “charge state purify” the precursor ion (prior to fragmentation) or the product ions (the result of fragmentation) greatly enhances the likelihood of a correct protein identification. Such methods are known in the art and generally rely on ion/ion transfer reactions to reduce multiply charged forms of the ion into lower charge states, preferably a single charged form. These methods include double isolation protocols as well as ion parking, as described above and also in the Example. Charge state purification greatly simplifies the interpretation of a product ion spectrum.

Dissociation/fragmentation of the mass selected precursor ions into a multiplicity of product ions can be accomplished using any convenient means, including but not limited to collision induced dissociation (CID) involving a gaseous target, photodissociation with UV or IR photons and surface induced dissociation. Any product ion can be analyzed, however typical product ions include b- and y-type ions and/or c- and z-type ions and/or, in the case of a negatively charged parent ion, a z-type ion missing a sulfur atom (a z-S ion). The type of product ion may be dependent on the dissociation method used; for example, electron capture dissociation will yield c and z-type ions, as opposed to b and y-type ions. Processing of the protein database(s) and scoring hits based on ion abundances are preferably tailored to the activation method used.

The masses of the product ions must, of course, ultimately be matched with data in a protein database in order to identify the protein which was dissociated in the mass spectrometer. The protein database can be, for example, an empirical protein database or a genomic database. An empirical protein database contains protein sequence information as well as, typically, annotations concerning structural variants or known processing events. Examples of empirical protein databases include SwissProt, trEMBL and the protein information resource (PIR). Unannotated databases, such as the NCBI_non-redundant database, can also be used.

Alternatively or in addition, the protein database can be a genomic (translational) database, such as GenBank. A genomic database is generated from the translated open reading frame (ORF) predictions from a partially or fully sequenced genome of an organism of interest, such as E. coli. K-12 strain MG 1655 in the Example below, or humans. The translated ORFs will contain many possible proteins. There is no limit on how many databases can be searched.

The information present in current genomic and empirical protein databases is generally not directly formatted for searching against protein mass spectrometry data. Since the method of the invention matches uninterpreted (i.e., lacking in assigned structural information) mass spectra with information obtained from the protein database(s), database information must be converted to mass information prior to making the comparison. Therefore, some degree of database manipulation or “pre-processing” is usually necessary or desirable. Furthermore, no current databases are “complete” with respect to inclusion of all mature proteins expressed by an organism, such as the identity and location of post-translational modifications. It is therefore desirable to expand the databases to include common or likely gene products formed from co- or post-translational processing, to the extent possible.

Accordingly, during the pre-processing phase, to begin with the database is typically properly formatted to enable or facilitate its interrogation by the search program. Next, it is often desirable to expand the database to account for one or more types of database annotations (i.e., known structural modifications) and/or one or more commonly observed, predicted or suspected post-translational modifications and/or, in the case of genomic databases, post-transcriptional modifications. For example in the case of bacterial proteins, the translated sequences typically begin with an initiation methionine as the N-terminal residue. However, approximately 50% of expressed bacterial proteins lack this initiation methionine. Thus, the genomic database is preferably customized to account for this common post-translational modification by including second entry for each ORF to allow for the possibility of N-terminal initiation methionine cleavage from each protein, effectively doubling the number of entries. While some of these proteins would not be observed due to the activity of the methionine aminopeptidase responsible for cleavage of the N-terminal methionine residue, as a function of the next adjacent amino acid, all of the processed sequences are preferably included in the database search procedure (see Link et al., Electrophoresis, 1997, 18, 1259-1313).

Besides N-terminal methionine, it may be desirable to expand the database to reflect other commonly observed structural modifications including, for example, the removal of signal sequences and propeptides, known or possible derivatizations such as glycosylation, phosphorylation or lipidation, methylation, acetylation, disulfide bond formation, translations of other reading frames or antisense sequences, predicted differential mRNA processing, or the presence of multiple protein chains in the one database entry.

If desired, all protein sequences in a database can be included in the comparison set which is processed and used to compare with the experimentally determined product ion masses. The comparison set likewise may include, at the discretion of the researcher, subsequences of protein sequence representing portions or fragments of a protein. Subsequences are typically selected using a user specified mass tolerance based on the mass of the protein of interest, as described in more detail below. Generally, when the term “database protein sequence” is used herein to describe a member of a comparison set, that term should be understood to include, at the option of the user, user-defined subsequences that are wholly within the database sequences.

It may be desirable to define a smaller comparison set by selecting one or more subsets of sequences from a larger database on the basis of constraints such as species or experimental mass of the parent protein as determined from the mass of the precursor ion. When limited by mass, this means selecting those database sequences that are characterized by mass that falls within a tolerance defined by the user. Mass tolerances for use in identifying members of a comparison set of database protein sequences (or subsequences of database protein sequences, representing fragments of proteins) based on the mass of the parent ion are typically no greater than one third of the parent ion mass. When a more narrowly defined parent ion mass window is desired, the mass tolerance can be defined by a narrower range of masses, for example, ±20 Da or even ±5 Da. Use of a larger mass tolerance maximizes the probability of accounting for post-translational modifications, thereby limiting the potential for false negatives. Use of a smaller mass tolerance minimizes the possibility for false positives by reducing the number of proteins subjected to search. It can be helpful to analyze the product ion spectra using more than one mass-limited database sequence subset, such as one with an intentionally large mass tolerance and one with an intentionally small mass tolerance.

The use of mass-limited database sequence subsets can lead to significant time savings compared to searching a complete database (i.e., where the entire database functions as the comparison set). The parent mass constraint can also add to the specificity of the approach. However, this approach is in general prone to “false negatives” since the mass of the mature (observed) protein may not correspond to the database entry. This is particularly problematic with the genomic databases but it is also of concern for the annotated empirical protein databases, which suffer from varying degrees of incompleteness.

It should be noted that when experimental parent mass is used to select a smaller comparison set from the universe of database proteins, the experimental mass can be matched to the mass calculated for the entire database sequence. As alluded to above, however, in a useful variation of this process experimental parent mass can also be matched to the mass of fragments of larger database proteins by analyzing protein subsequences. The mass of sequence fragments can be conveniently determined using an internal sliding window and matched against the experimental mass and tolerance. Consecutive mass fragments are analyzed as this mass window moves along the protein sequence. The database is thereby processed using a parent mass constraint to define subsets of sequences from each protein entry for subsequent searching by successively calculating the masses of sequences within a larger chain. This approach is intended to find those cases in which the observed protein is a fragment of a putative protein, including fragments in which regions of each end of the putative protein are missing. This approach is significantly more time intensive because the number of sequences to be searched is much higher.

As described below in the Example and noted above, it is also certainly possible to search an entire protein database against the experimentally observed product ion masses, using a predetermined mass uncertainty (i.e., mass tolerance) for the product ions only, i.e., without constraints placed on the parent mass. In this embodiment of the method, the entire database functions as the comparison set. This is a slower process and, since many more proteins are being searched, the likelihood for a “false positive” is higher than with the parent ion mass constraint. However, there is a lower “false negative” rate because the mass of the experimentally observed proteins that differ significantly in mass from the predicted values may still be matched with the predicted sequence, as long as error tolerant search methods are used. The difference between the observed and predicted parent ion masses provides information on the origin of the discrepancy between the putative and observed protein.

Once a set of sequences has been selected from a processed database to form the comparison set (or the decision has been made to use the entire database as a comparison set), the sequence information for the proteins and protein fragments, if any, in the comparison set must be converted to predicted product ion mass information via in silico fragmentation to allow for matching against the experimentally determined protein product ion masses. The comparison can be based on either absolute product ion masses, or relational product ion masses.

For multiply-protonated proteins, generation of an absolute product ion mass list from the database information typically entails generating mass lists corresponding to the possible b- and y-type ions for each sequence in the comparison set. However, the method is not limited to the generation of any particular product ion type, and any possible subset of fragment ion masses can be determined, as desired, for matching against experimental data. For example, in the case of deprotonated proteins that have disulfide linkages, the calculation of masses of the complementary z-S and c-type ions arising from cleavages at disulfide linkages may be useful for protein identification. Regardless of the fragment ion type, the absolute masses of the product ions have a direct relationship to either the N- or C-terminal end of the sequence under examination.

Instead of using the absolute mass of the product ions, protein identification/characterization can be approached by matching differences in product ion masses (relative or relational masses). For example, the differences in the masses of product ions derived from user specified cleavage sites (such as preferential or “favored” cleavages at Pro, Asp, Lys, etc., as described below) can be used to generate a relational product ion mass list. Note that the relational mass approach requires processing of both the database and the mass spectral data. Searches based on relational mass differences are expected to be most valuable when the analyzed protein is not accurately reflected in the database (e.g., when post-translational modifications not reflected in the database are present).

In both these approaches, product ion mass spectral data (mass) is compared to the calculated masses for the predicted product ions produced by in silico fragmentation proteins in the comparison set derived from the database(s), preferably using a defined mass tolerance. The mass tolerance is instrument-dependent; it depends upon the mass measurement accuracy of the mass spectrometer. Tolerance can be in terms of Daltons (Da) or parts per million (ppm) for an ion trap mass spectrometer such as the one used in the Example. For an ion trap quadropole tandem mass spectrometer, a mass tolerance of about 100 ppm is usually attainable; for time-of-flight mass spectrometers, the mass tolerance is typically around 10 ppm or less, such as 1 ppm; and for a good Fourier transform ion cyclotron resonance instrument, a mass tolerance of around 1 ppm is possible. The results must then be scored for each protein in the comparison set. The least complicated means for ranking possible matches is a simple count of the number of experimentally determined product ion masses (relational or absolute) that match calculated masses derived from the database, within a specified mass tolerance.

In many cases, however, it is desirable to provide a greater degree of discrimination than that obtained with a simple count of matches. A means to do so, which is justified on physical grounds, is to apply weighting factors to the matches to reflect known biases in product ion formation. For example, particular amino acids tend to give rise to favored cleavages. A “favored cleavage site” indicates a peptide bond that cleaves more frequently than other peptide bonds under the particular conditions used to dissociate the parent protein precursor ion. For example, for positively charged precursor ions, fragmentation is generally more likely to occur at N-terminal proline, C-terminal aspartate and glutamate, and C-terminal lysine. Product ion fragments resulting from cleavage at the favored sites can be specified by the user and weighted more heavily in the scoring scheme. Matches can also be weighted on the basis of product ion type (e.g., the expected prevalence of c and z- or z-S ion fragments vis a vis b- and y-type ion fragments).

Moreover, experience with charge state dependent protein ion fragmentation shows that the values of these weighting factors can be influenced by parent ion charge state and/or parent ion charge polarity. Both may affect which cleavage sites are favored, and parent ion charge polarity may affect the type of product ions produced as well. For example, weighting factors for N-terminal Pro are highest at relatively high parent ion charge states, and weighting factors for C-terminal Asp cleavages highest at intermediate to low parent ion charge states. An example relating to parent ion polarity is weighting c and z-S fragments more heavily than other product ions for negatively charged proteins. For negatively charged parent ions, the database can, for example, be processed to match against c and z-S type ions at cysteine residues only.

Product ion abundance is another factor used in the scoring algorithm according to the invention. Product ion abundance is taken as either the height or the area of a peak in the product ion spectrum and is usually normalized to the most abundant peak. For example, the most abundant product ion is assigned an abundance of 100 and all others are assigned according to this scale. The heaviest weight is given to those channels that give rise to the greatest extent of fragmentation.

An illustrative scoring algorithm for multiply protonated (i.e., positively charged) protein parent ions that fragment to give b- and y-type ion is shown below:
Score=C_{P}([ΣI]nP)+C_{D}([ΣI]nD)+C_{K}([ΣI]nK)+C_{E}([ΣI]nE)+C_{X}([ΣI]nX)
where ΣI is the sum of intensities of the product ions corresponding to each fragmentation type, expressed as a percent fraction of the normalized total product ion abundance; nP, nD, nK, nE and nX are the number of product ions observed corresponding to cleavages at the N-terminal of proline (P), the C-terminal of aspartic acid (D), lysine (K) and glutamic acid (E), or at any other “non-specific” residues (X), respectively (i.e., cleavages at all other residues); and C_{P}, C_{D}, C_{K}, C_{E} and C^{X} are user-defined coefficients to weight for the cleavages corresponding to the known preferential fragmentation sites. The inclusion in the scoring algorithm of the abundances of each product ion, expressed as a percent fraction of the normalized total product ion abundance gives greater weight to those cleavages that inherently yield abundant product ions.

### EXAMPLE

The present invention is illustrated by the following example. It is to be understood that the particular examples, materials, amounts, and procedures are to be interpreted broadly in accordance with the scope and spirit of the invention as set forth herein.

### Gas-Phase Concentration, Purification and Identification of Whole Proteins from Complex Mixtures

In this example, we demonstrate that the ion parking approach can be used to facilitate the gas-phase concentration and purification of selected protein ions from a complex protein mixture for subsequent dissociation in a quadrupole ion trap mass spectrometer. Five proteins present in a relatively complex mixture derived from a whole cell lysate fraction of E. coli containing about 30 components were concentrated, purified and dissociated in the gas-phase, using a quadrupole ion trap mass spectrometer. Concentration of intact protein ions was effected using gas-phase ion/ion proton transfer reactions in conjunction with mass-to-charge dependent ion “parking” to accumulate protein ions initially dispersed over a range of charge states into a single lower charge state. Sequential ion isolation events interspersed with additional ion parking ion/ion reaction periods were used to “charge state purify” the protein ion of interest. Five of the most abundant protein components present in the mixture were subjected to this concentration/purification procedure then dissociated by collisional activation of their intact multiply charged precursor ions.

Additionally, we demonstrate that database interrogation of the uninterpreted mass spectrometry-derived whole protein ion data allows unambiguous identification of these proteins. Four of the five proteins, ranging in mass from 7 to 10 kDa, were subsequently identified by matching the uninterpreted product ion spectra against a partially annotated protein sequence database, coupled with a novel scoring scheme weighted for the relative abundances of the experimentally observed product ions and the frequency of fragmentations occurring at preferential cleavage sites.

The identification of these proteins illustrates the potential of this “top down” protein identification approach to reduce the reliance on condensed-phase chemistries and extensive separations for complex protein mixture analysis.

Materials and Methods

Materials. Acetic acid and acetonitrile were obtained from Mallinckrodt (Paris, Ky.). Trifluoroacetic acid (TFA) was purchased from Pierce (Rockford, Ill.). Glucose, CaCl₂, thiamine and NaCl were from Sigma (St. Louis, Mo.). Tryptone and yeast extract were obtained from Fisher Scientific (Pittsburg, Pa.). Agar was purchased from DIFCO (Sparks, Md.).

Growth and lysis of E. coli. Freeze dried ATCC 15597 E. coli was obtained from American Type Culture Collection (Rockville, Md.) and reactivated on agar plates at 37° C. for 24 hours under sterile conditions. The media used to prepare the agar plates and grow the E. coli was composed of 10 mL of 10% glucose, 2.0 mL of 1M CaCl₂, 1.0 mL of 10 mg/mL thiamine, 10 g tryptone, 1.0 g yeast extract, and 8.0 g NaCl, per liter. The plates used for plating contained the same ingredients plus 10 g agar per liter. Reactivated E. coli colonies were removed from the agar plates and suspended in 100 mL of growth media in 250 mL culture flasks. Aerobic growth was carried out at 37° C. until the media reached an optical density of 2.0 at 600 nm. The E. coli was then harvested by centrifugation at 3,000 g for 10 min and resuspended in 10 mL water plus 1 mL of protease inhibitor (Calbiochem, San Diego, Calif.). Lysate was prepared by subjecting this mixture to intense bursts of ultrasonic power while using an ice bath to minimize heating. The lysate was then centrifuged at 5,000 g for 20 minutes to remove any remaining fragments of E. coli and the soluble lysate fraction was stored at −70° C. until required.

Fractionation of proteins from the soluble E. coli whole cell lysate by RP-HPLC. Proteins from the soluble whole cell lysate of E. coli (150 μL/10 mL total) were fractionated by reversed-phase HPLC on a Hewlett Packard (Palo Alto, Calif.) model 1090 HPLC, using a Poros (Applied Biosystems, Foster City, Calif.) R1/10 100 mm×2.1 mm I.D. column operated at 0.5 mL/min. A linear 12 minute gradient from 0 to 100% B was used, where buffer A was 0.1% aq. trifluoroacetic acid (TFA) and buffer B was 60% acetonitrile/40% H₂O containing 0.09% TFA. The column was maintained at a constant temperature of 40° C. The absorbance was monitored at 215 nm and fractions were collected at 0.5 minute intervals. The collected fractions were lyophilized to dryness then dissolved in 250 μL of 1% aqueous acetic acid prior to introduction to the mass spectrometer. Based on the UV response of the HPLC fraction, it is estimated that 1-5 pmol of each of the proteins subjected to MS/MS were loaded into the nanospray tube.

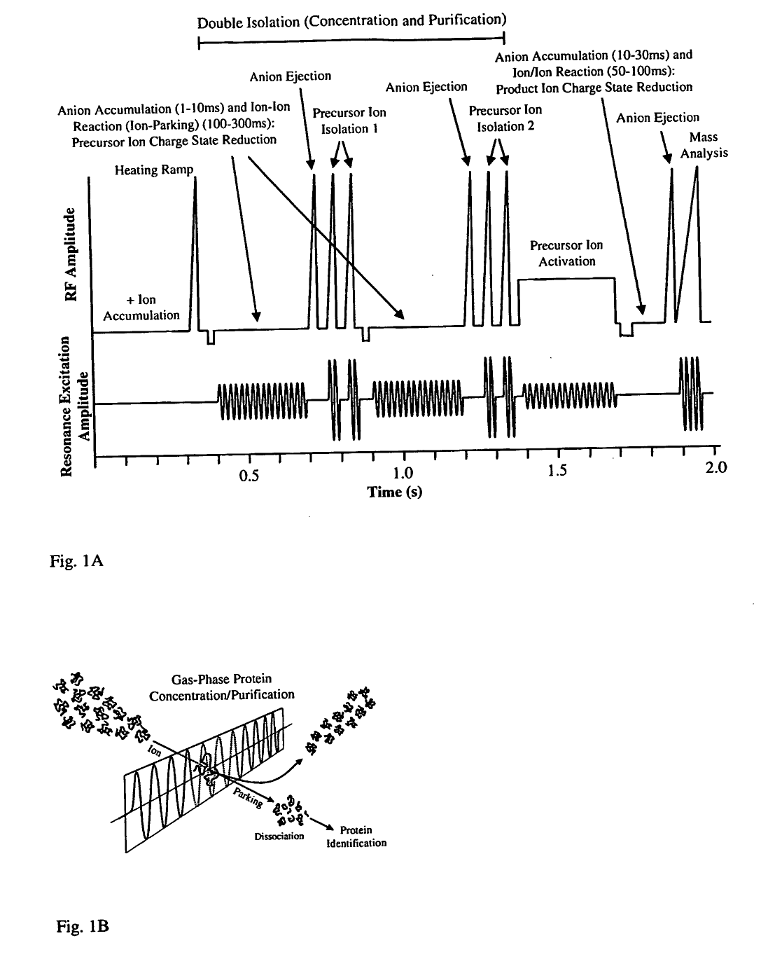

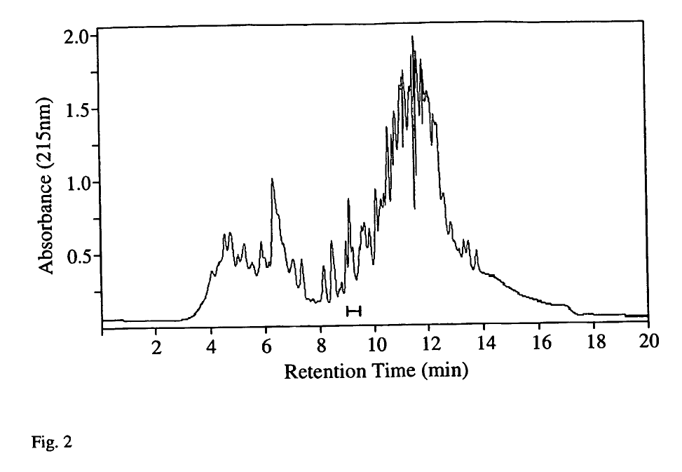

Mass Spectrometry. A Finnigan ITMS quadrupole ion trap, modified for ion introduction through the entrance end cap electrode by electrospray ionization, and via the ring electrode for atmospheric sampling glow discharge ionization, has been described previously (Stephenson et al., Int. J. Mass Spectrom. Ion Proc. 1997, 162, 89-106). Solutions (10 μL) were introduced to the mass spectrometer by infusion at a flow rate of approximately 20 to 40 nL/min using a home-built nanospray ion source. Briefly, nanospray tips were produced from 1.5 mm O.D.×0.86 mm I.D. borosilicate glass capillaries using a Sutter Instruments model P-87 micropipette puller (Novato, Calif.) held in place during operation by a Warner Instruments (Hamden, Conn.) E series microelectrode holder. The electrical connection to the solution (typically 1.0-1.2 kV) was made by inserting a stainless steel wire through the back of the capillary. In a typical experiment, (see FIG. 1), after an electrospray ion accumulation period of several hundred milliseconds, a “heating ramp” was performed to collisionally remove weakly bound non-covalent adducts by applying a low amplitude single frequency resonance excitation voltage to the end caps while simultaneously sweeping the amplitude of the RF applied to the ring electrode.

For lower abundance proteins present in the mixture, ion parking was then performed by applying a single frequency resonance excitation voltage approximately 200 Hz lower than the fundamental secular frequency of motion of a selected m/z region of interest, while subjecting the total ion population to ion/ion proton transfer reactions with the singly charged [M-F]⁻ and [M-CF₃]⁻ anions derived from glow discharge ionization (McLuckey et al., Anal. Chem. 1988, 60, 2220-2227) of perfluoro-1,3-dimethylcyclohexane (PDCH). The effect of this ion parking voltage is to concentrate all the higher charge states initially present in the mass spectrum of a selected protein into a single lower charge state at the m/z of interest (McLuckey et al., Anal. Chem. 2002. 74, 336-346). A 10 ms ramp of the RF amplitude was then used to eject residual PDCH anions in order to avoid deleterious effects during further isolation or mass analysis (Stephenson et al., Anal. Chem. 1997, 69, 3760-3766). Isolation of ions in the specified m/z range was then performed using multiple resonance ejection ramps to sequentially eject ions of m/z higher and lower than that of interest (McLuckey et al., J. Am. Soc. Mass Spectrom. 1991, 2, 11-21).

Following isolation of this initial m/z range, lower charge state precursors were formed by subjecting the isolated ion population to an additional ion/ion proton transfer reaction period. Further concentration and “charge state purification” was performed by applying an ion parking voltage at a second m/z of interest, corresponding to a lower charge state of the selected protein, during the ion/ion reaction.

Following ejection of residual PDCH anions and further isolation of the m/z region of interest containing the concentrated and purified protein precursor ion charge state, collision induced dissociation (CID) was performed by applying a single frequency resonance excitation voltage corresponding to the center of mass of the ion of interest to the end caps, ranging from 200 to 400 mVₚₚ, for 300 ms. CID conditions were optimized to maximize the product ion signal to noise ratio. A final ion/ion reaction period was then employed to reduce the multiply charged product ion population to predominantly their singly charged forms, thereby simplifying their interpretation.

Following ejection of residual PDCH anions, a product ion spectrum was then acquired by resonance ejection (Kaiser et al., Int. J. Mass Spectrom. Ion Proc. 1991, 106, 79-115). The spectra shown are the average of 300-500 individual mass analysis scans. Calibration of the pre- and post-ion/ion product ion mass spectra was performed using the singly, doubly and triply charged ions of either bovine cytochrome c or bovine ubiquitin formed by ion/ion reactions in the absence of collisional activation.

Protein Identification by Database Searching of the Uninterpreted Whole Protein

MS/MS spectra. Protein identification via interrogation of the post-ion/ion reaction product ion spectra was performed using a program written using Active Perl for Windows (v. 5.6.0.616) for use on a Windows OS (Microsoft) computer. The databases used were: i) the translated open reading frame (ORF) predictions (4290 possible proteins) in FASTA format of the E. coli. K-12 strain MG1655 (version M52) genome as sequenced by the E. coli Genome Project at the University of Wisconsin-Madison (Blattner et al., Science. 1997, 277, 1453-1474), modified to include a second entry for each ORF to allow for the possibility of N-terminal initiation methionine cleavage from each protein (Gonzales et al., FEMS Microbiol. Rev. 1996, 18, 319-344), and; ii) the entries corresponding to E. coli (4736 entries) extracted from the SWISS-PROT protein sequence database (Bairoch et al., Nucleic Acids Res. 2000, 28, 45-48) (release 40.0). Further processing of the SWISS-PROT entries was then performed by interrogation of the feature table (FT) line in each entry to account for known annotations, such as the removal of signal sequences and propeptides and the possibility of multiple protein chains being present in the one database entry. In addition, the possibility for N-terminal initiation methionine cleavage from each of the processed entries was also taken into account. This processing yielded a database of 8598 entries.

To search the databases against the experimentally derived data, each protein in the database matching the experimentally determined protein precursor ion mass within a specified mass tolerance range (±10 Da) was retrieved and the masses of the predicted b- and y-type fragment ions for each entry were compared to a user defined list of experimentally derived product ion mass values, with a specified fragment ion mass tolerance of ±5 Da. The results were then ranked according to the number of matches. A score was then applied to each result, using the equation shown below,
Score=5([ΣI]nP)+5([ΣI]nD)+4([ΣI]nK)+2([ΣI]nE)+([ΣI]nX)
where ΣI is the sum of intensities of the product ions corresponding to each fragmentation type, expressed as a percent fraction of the normalized total product ion abundance, and nP, nD, nK, nE and nX are the number of product ions observed corresponding to cleavages at the N-terminal of proline, the C-terminal of aspartic acid, lysine and glutamic acid, or at any other “non-specific” residues, respectively. This scoring approach has some differences from those developed previously (Li et al., Anal. Chem. 1999, 71, 4397-4402; Meng et al., Nat. Biotechnol. 2001, 19, 952-957). Here, in addition to weighting the score to account for cleavages corresponding to known preferential fragmentation sites (5 times for cleavage N-terminal to proline or C-terminal to aspartic acid, 4 times for cleavage C-terminal to lysine, and 2 times for cleavage C-terminal to glutamic acid), we have also included the abundances of each product ion, expressed as a percent fraction of the normalized total product ion abundance, in the scoring algorithm, thereby giving greater weight to those cleavages that inherently yield abundant product ions.

Results and Discussion

Gas-Phase Concentration, Purification and Dissociation of Selected Protein Precursor Ions. In order for the top down strategy to be effective for generating interpretable sequence information from whole protein ions present in complex mixtures, the precursor ion selected for dissociation and subsequent identification must be efficiently isolated from other ions present in the mixture, prior to its dissociation. To demonstrate the efficacy of the ion parking technique for gas-phase protein purification and concentration, the identification of proteins from the soluble protein fraction derived from a whole cell lysate of E. coli was performed in this study. 4290 proteins are predicted from the translated open reading frames of the fully sequenced E. coli genome. It is expected that the expression of many of these at any given time, as well as the presence of any post-translational modifications, will result in a very complex protein mixture. Indeed, it has been suggested that each ORF produces on average 1.4 proteins, potentially resulting in over 6000 proteins (Tonella et al, Proteomics 2001, 1, 409-423). Previously, using multiple narrow pH range 2D gels, it has been estimated that over 70% of the proteome can be visualized at any given time (Tonella et al, Proteomics 2001, 1, 409-423).

The mass spectrum obtained following ESI-MS and ion/ion reactions of the crude soluble whole cell lysate was characterized by an elevated baseline of chemical noise ranging from m/z 1000 to 30000, with few clearly distinct peaks, reflecting the extreme complexity of the mixture (data not shown). To partially simplify this complex mixture and allow further analysis by mass spectrometry, a portion of the whole cell lysate (150 μL/10 mL total) was loaded onto a Poros R1/10 100 mm×2.1 mm I.D. column (reverse phase high pressure liquid chromatography, RP-HPLC) and developed at 0.5 mL/min using a 12 minute linear gradient as described in above. The absorbance was monitored at 215 nm and fractions were collected at 0.5 min intervals. Fractions corresponding to retention times 3 to 8 minutes were found to contain mainly low molecular weight (<2 kDa) species and hence were not examined further here. Fractions from retention times 8 to 16 minutes were found to contain up to 200 proteins per fraction, ranging in mass from 5000 to 60000 Da. (Note that the maximum observable mass range for singly charged protein ions in the current instrumentation is 66,000 Da).

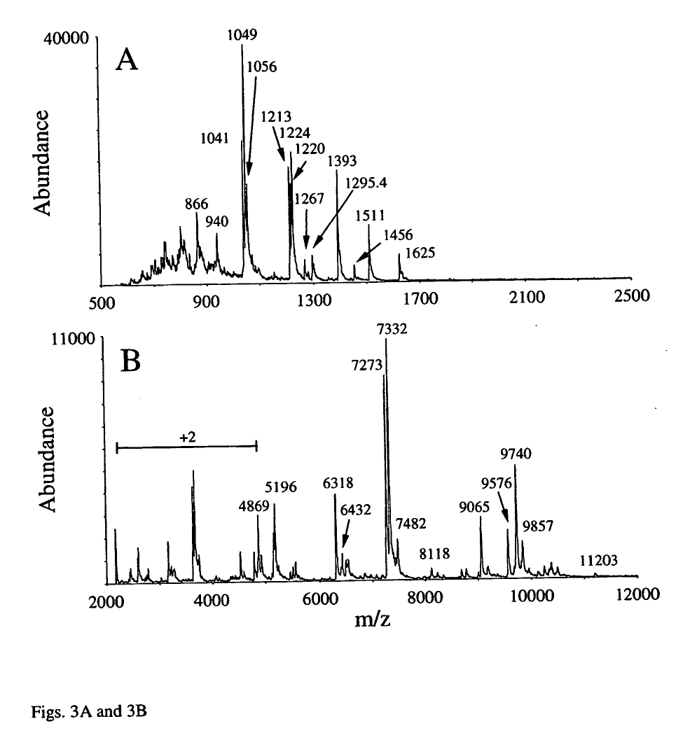

For protein mixtures of this complexity, the utility of ion/ion reactions for charge state reduction and subsequent interpretation of the spectra is clearly apparent. For example, analysis of the protein fraction corresponding to retention times 9.0-9.5 minutes, in the absence of ion/ion reactions (i.e., the pre-ion mass spectrum), resulted in the spectrum shown in FIG. 3A. The pre-ion/ion mass spectrum was acquired using a resonance ejection frequency of 89202 Hz at an amplitude of 10.5 V. While the masses of the most abundant proteins present in this mixture could be determined from their charge state distributions (ions ranging from m/z 1000 to 1600), the complicated array of ions in the range of m/z 600 to 1000, corresponding to overlapping charge state distributions of low abundance proteins present in the mixture, make determination of the masses of these components problematic. In contrast, determination of the masses of the individual components making up the mixture, including those at low abundance, was readily achieved after subjecting the initial multiply charged ion population to ion/ion proton transfer reactions with singly charged anions. The post-ion/ion reaction mass spectrum (FIG. 3B) was acquired at 17000 Hz and an amplitude of 1.7 V following ion/ion reactions using anion accumulation and ion/ion mutual storage times of 30 and 100 ms, respectively. The resultant post-ion/ion reaction mass spectrum was found to contain predominantly singly charged ions from which up to 30 proteins, ranging in mass from 5000 to 11000 Da, could be observed. Note that the doubly charged ions can be identified from both their mass-to-charge ratios (one-half those of the singly-charged ions) and their abundance ratios, which mirror the ratios of the corresponding singly-charged ions.

The region surrounding the most abundant ion in the mixture at m/z 1049, containing at least three major ions, was isolated using multiple resonance ejection ramps. Following isolation, this m/z region was concentrated and purified by ion parking. The selected m/z region was first subjected to a short ion/ion reaction period to “charge state purify” the various components present.

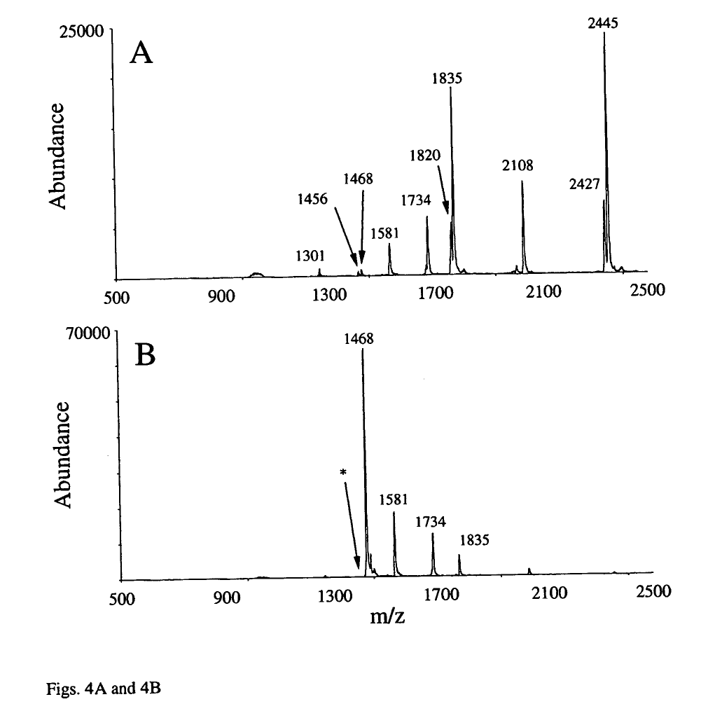

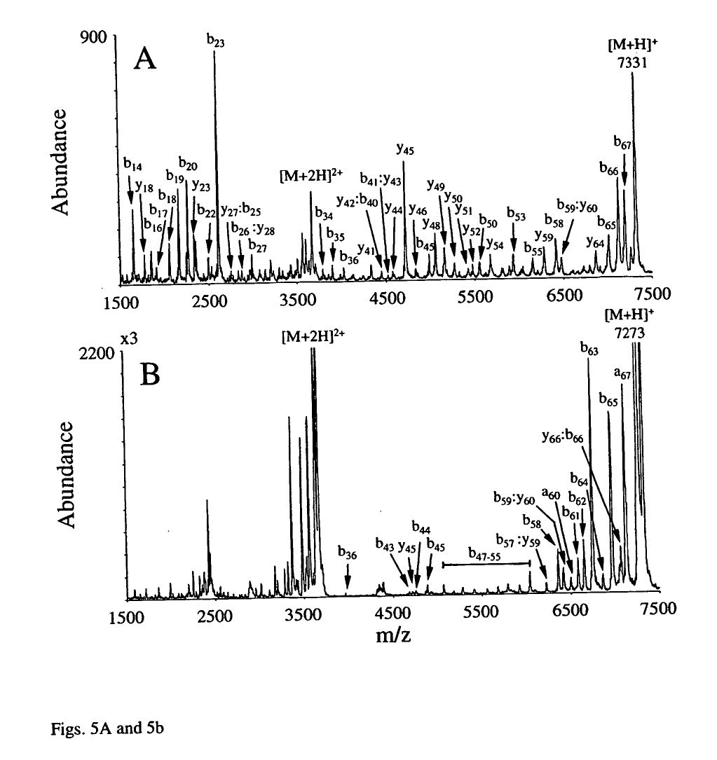

The post-ion/ion reaction mass spectrum of this isolated m/z 1049 (±10 Da) region, acquired after a 2.5 millisecond anion accumulation period and a 300 millisecond mutual ion storage period is shown in FIG. 4A.

From this data, it can be seen that there were actually four major components present in the initially isolated m/z region; m/z 2445(+3), 1835(+4) and 1468(+5), corresponding to the protein at mass 7332 in FIG. 3B, m/z 2427(+3), 1820(+4) and 1456(+5), corresponding to the protein at mass 7273 in FIG. 3B, m/z 2108 (+3) and 1581(+4), corresponding to the protein at mass 6318 in FIG. 3B, and m/z 1734(+3) and 1301(+4), corresponding to the protein at mass 5196 in FIG. 3B.

Using identical anion accumulation and ion/ion reaction conditions, an ion parking voltage was then applied during the ion/ion reaction period to concentrate all the ion current associated with the most abundant protein (mass 7332 in FIG. 3B) into its +5 charge state (m/z 1468) (FIG. 4B). Spectra were acquired using the same resonance ejection conditions as described in FIG. 3A. By placing the frequency of the ion parking resonance excitation voltage on the low mass (high frequency) side of the ion of interest during the ion/ion reaction period (frequency 25300 kHz, amplitude of 1.0V), ions corresponding to the protein at mass 7273 were ejected from the ion trap as they passed through their +5 charge state, as the m/z of this ion (m/z 1456) falls directly on-resonance with the applied ion parking voltage. Thus, ions corresponding to this protein are absent in the post-ion/ion mass spectrum shown in FIG. 4B. The charge states of the two other proteins initially present in the m/z 1049 region do not have m/z values close to the frequency of the applied ion parking voltage so are not substantially affected.

Following isolation of the parked +5 charge state (m/z 1468) of the protein at mass 7332, CID was then performed. FIG. 5A shows the post-ion/ion reaction CID MS/MS spectra of the [M+5H]⁵⁺ ion (m/z 1468 in FIG. 4B) of the protein at mass 7332 in FIG. 3B. This spectrum was obtained after reducing the multiply charged product ions to primarily their singly charged forms by an additional ion/ion reaction period. Precursor ion activation conditions were 88725 Hz and 240 mV for 300 ms. Post-CID anion accumulation and ion/ion reaction times were 25 ms and 100 ms, respectively. The spectrum was acquired at 25500 Hz and an amplitude of 1.7 V.

In a separate experiment, using identical initial isolation conditions, the ion current associated with the next most abundant protein in this fraction (mass 7273 in FIG. 3B) was concentrated by ion parking of its +5 charge state (m/z 1456) via the application of an ion parking voltage on the high mass side of the ion. Following re-isolation, this ion was subjected to CID and ion/ion reactions to produce the post-ion/ion MS/MS spectrum shown in FIG. 5B. Post-CID anion accumulation and ion/ion reaction times were 15 ms and 90 ms, respectively. The spectrum was acquired at 25000 Hz and 1.7 V. Note that a small amount of the singly charged ion corresponding to the protein at mass 7332 can be observed in this spectrum, due to incomplete isolation of its +5 charge state from the desired precursor ion prior to CID and ion/ion reactions. However, this ion was not activated by the single frequency resonance excitation voltage applied during the CID experiment and so did not contribute product ions to the spectrum.

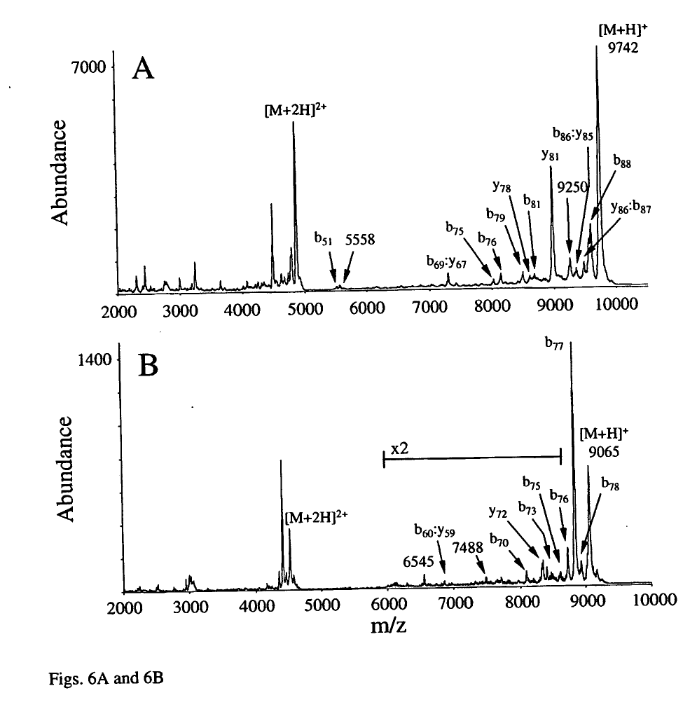

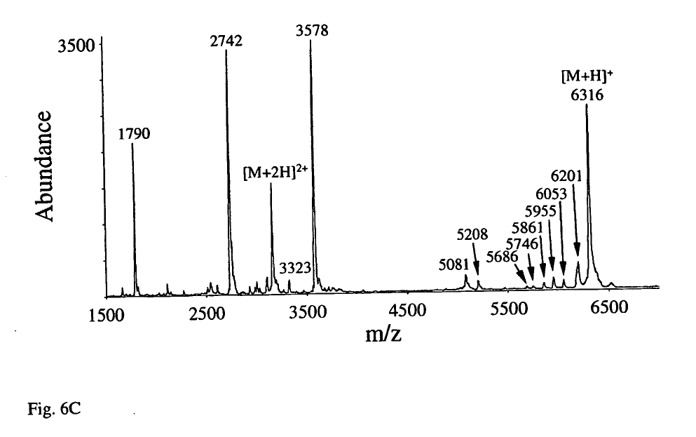

Fragmentation of the lower abundance proteins at masses 9740, 9065 and 6318 in the fraction was also effected after concentration and purification of their [M+7H]⁷⁺, [M+6H]⁶⁺ and [M+5H]⁵⁺ charge states, respectively, by ion parking. In these cases, an initial ion parking ion/ion reaction period was employed prior to the first isolation step to accumulate all the ion current corresponding to higher charge states of the protein of interest into the charge state one higher than that eventually subjected to CID, thereby increasing the sensitivity for these lower abundance proteins. Their post-ion/ion reaction MS/MS spectra are shown in FIGS. 6A-C, respectively. The 1 to 2 Da mass differences observed between the MS and MS/MS data for these proteins are most likely due to variations in the number of ions between the two spectra, causing subtle changes in their masses relative to the mass calibration.

Identification of unknown proteins by database searching of uninterpreted post-ion/ion reaction MS/MS spectra. A program was written in-house to identify unknown proteins by database interrogation of their uninterpreted post-ion/ion reaction MS/MS spectra. Initially, a database was generated from the translated open reading frame (ORF) predictions from the fully sequenced genome of E. coli. K-12 strain MG1655. The translated ORFs of the E. coli genomic sequence contain 4290 possible proteins, all with an initiation methionine as the N-terminal residue. However, approximately 50% of expressed bacterial proteins lack this initiation methionine. To account for this common post-translational modification, a second entry for each ORF was included to allow for the possibility of N-terminal initiation methionine cleavage from each protein, giving a total of 8580 entries. While some of these proteins should not be observed due to the activity of the methionine aminopeptidase responsible for cleavage of the N-terminal methionine residue, as a function of the next adjacent amino acid (Gonzales et al., FEMS Microbiol. Rev. 1996, 18, 319-344), all of the processed sequences were included in the database search procedure as a previous report from Link et. al. indicated that many of the expressed proteins do not follow these cleavage rules (Link et al., Electrophoresis. 1997, 18, 1259-1313).

Database searching was initiated by retrieving from the specified database a list of candidate proteins matching the experimentally determined protein precursor ion mass within a specified mass tolerance range (±10 Da). Then, the masses of the predicted b- and y-type fragment ions for each protein were compared to a user-defined list of experimentally derived product ion mass values, with a specified fragment ion mass tolerance of ±5 Da The results were then ranked according to the number of matches, scored, and output to a file for manual interrogation.

For the protein at mass 7332 (7331 in FIG. 5A), three proteins within the specified mass tolerance were initially retrieved from the database, (primary accession numbers P76571, P76106 and P36997 with calculated [M+H]+ ion masses of 7331.4, 7331.5 and 7333.3 Da), where the masses of each of the retrieved proteins corresponded to entries lacking the initiating methionine. The predicted b- and y-type product ions for all of the retrieved proteins were then compared to 60 product ion masses obtained from the data shown in FIG. 5A. The top ranked protein, with 56 of 60 matching ions and a calculated score of 249.58, corresponded to cold shock-like protein E (CspE) with cleavage at 39 (57%) of the amide bonds along the protein backbone (Table 1). Five of the experimentally observed product ions matched within the database search tolerance of +5 Da, both b- and y-type ions predicted from the retrieved sequence (indicated by italics in Table 1), and were therefore counted twice by the scoring algorithm. These ions are labeled twice in the spectrum in FIG. 5A. Prediction of the likely identities of several of these may be made however, based on factors such as fragmentation at a favored site (e.g., an aspartic acid, proline, lysine or glutamic acid residue) or the appearance of one of the ions in a contiguous series of b- of y-type products ions. The second and third ranked proteins matched only 11 and 5 of the experimentally determined product ion masses with calculated scores of 13.89 and 10.85, respectively. Additionally, the matching product ions from these proteins do not correspond to any of the 10 most abundant product ions seen in FIG. 5A.

Six proteins were initially retrieved from the ORF database for the protein at mass 7273 (FIG. 5B). 27 of 29 experimentally observed product ion masses included in the database search were found to correspond to product ions derived from cold shock like protein C (CspC, accession number P36996) lacking the N-terminal methionine (see Table 1), with a calculated score of 137.25. The second and third ranked proteins (accession numbers P76136 and P15277) matched only 9 and 8 of the 29 product ion masses with scores of 43.37 and 35.37, respectively. Interestingly, the masses of two of the product ions not initially matched by the search routine to cold shock-like protein C closely corresponded to a-type ions (a₆₇ and a₆₀ in FIG. 5B), whose formation is not included in the search parameters. It is unclear at this time why the formation of these ions, not normally observed in the product ion spectra generated from the dissociation of whole proteins, were observed for this protein in relatively high abundance.

FIG. 6 shows post-ion/ion reaction CID MS/MS spectra for additional product ions shown in FIG. 3B. In particular, FIG. 6A shows the post-ion/ion reaction CID MS/MS spectrum of the [M+7H]⁷⁺ ion of the protein at mass 9740 in FIG. 3B. Precursor ion activation conditions were 88250 Hz and 215 mV for 300 ms. Post-CID anion accumulation and ion/ion reaction times were 20 ms and 100 ms, respectively, and the spectrum was acquired at 21000 Hz and 1.7 V. FIG. 6B shows the post-ion/ion reaction CID MS/MS spectra of the [M+6H]⁶⁺ ion of the protein at mass 9065 in FIG. 3B. Precursor ion activation conditions were 88300 Hz and 230 mV for 300 ms. Post-CID anion accumulation and ion/ion reaction times were 20 ms and 100 ms, respectively, and the spectrum was acquired at 22000 Hz and 1.7 V. Finally, FIG. 6C shows the post-ion/ion reaction CID MS/MS spectrum of the [M+5H]⁵⁺ ion of the protein at mass 6318 in FIG. 3B. Precursor ion activation conditions were 88100 Hz and 215 mV for 300 ms. Post-CID anion accumulation and ion/ion reaction times were 25 ms and 100 ms, respectively, and the spectrum was acquired at 25000 Hz and 1.7 V.

Searches of the translated ORF database for the three remaining proteins with masses of 9740, 9065 and 6318 (9742, 9065 and 6316 in FIGS. 6A-C, respectively), failed to result in positive identifications. Five entries in the database that matched to within ±10 Da of the protein at 9742 (FIG. 6A) were retrieved, however, only 5 of the 13 experimentally observed product ion masses matched predicted ions from the top ranked candidate (accession number P32693), with a calculated score of 63.27. Additionally, none of the three most abundant product ions in the experimentally observed data (m/z 8972, 9250 and 9592), corresponding to 61% of the total product ion abundance, were matched to any of the predicted products. Five entries were retrieved from the database for the protein at mass 9065 (FIG. 6B). While the top ranked protein (accession number P76358) matched 5 of 11 product ion masses, its calculated score was only 18.61 and the two highest abundance products (m/z 8835 and 8726) comprising 77.5% of the total product ion abundance were not matched to any of the predicted products. For the protein at mass 6316 (FIG. 6C), only one protein was retrieved from the database within the specific mass tolerance (accession number PO₂₄₃₅), however the predicted fragment ions only had a calculated score of 16.87 and failed to match either of the two most abundant product ions (m/z 3578 and 2742), comprising 74.8% of the total product ion abundance. Searches using wider precursor and product ion mass tolerances of ±20 Da and ±10 Da, respectively, retrieved greater numbers of proteins in each case, but did not result in positive identifications, indicating that the relatively low mass accuracies obtained using the current instrumentation were not the cause of the inability to identify these proteins.

To determine whether the inability to identify these proteins was due to the presence of additional post-translational processing events that were unaccounted for using the search procedure described above, we generated a custom database of E. coli sequences extracted from the SWISS-PROT protein sequence database (4736 entries). These entries were then processed, using the annotations listed in each entry, to account for known modifications such as the cleavage of initiating methionine, the removal of signal sequences and propeptides, or the presence of multiple protein chains in the one database entry. The possibility of N-terminal initiation methionine cleavage from these new entries was also included, yielding a database of 8598 proteins.

Searching this new database using the same product ion masses and identical search tolerances as described above, the two proteins at mass 7332 and 7273 were again confirmed as cold shock-like proteins E and C (FIGS. 5A and 5B), with identical product ion matches and scores as those resulting from the translated ORF database search. Four proteins were initially retrieved from the modified SWISS-PROT database with calculated masses of 7332±10 Da. The second ranked protein in this case (accession numbers P23518), matching 14 of 60 product ions and with a score of 56.03, does not appear in the translated ORF database so was not listed during the earlier search procedure. Seven proteins were retrieved from the modified SWISS-PROT database for the protein at mass 7273. The second ranked protein listed here (accession number P15277), matching 8 of the 29 product ion masses and a score of 35.37, was ranked third in the translated ORF database search. Closer inspection reveals that the second ranked protein from the ORF database is not present in the SWISS-PROT database.

More importantly, the proteins with masses of 9740 and 9065 (9742 and 9065 in FIGS. 6A and 6B, respectively), which were not identified by the earlier search procedure, were positively identified here using the modified SWISS-PROT database search approach. The protein at mass 9742 was found to correspond to protein hdeA precursor (accession number P26604), with removal of the signal peptide consisting of the first 21 residues of the amino acid sequence. 14 of 13 product ion masses were matched (three of the product ion masses each matched two predicted fragments within the search tolerances, while two product ions were not matched) with a calculated score of 449.35. The second ranked protein from this search (accession number P32693) was listed previously as the top ranked protein in the ORF database search results. The protein at mass 9065 was identified as protein hdeB precursor (accession number P26605; 9 of 11 matching product ion masses and a score of 429.16) with removal of the signal peptide of 29 residues from the N-terminus. Here, the second ranked protein (accession number PO₂₄₃₅) matched only 5 of 19 masses with a score of 16.87.

While three proteins were retrieved from the modified SWISS-PROT database for the protein at mass 6316, the top ranked candidate (5 of 19 matches) again corresponded to a protein with accession number P02435, as was observed from the ORF database search. Therefore, the correct identity of this protein was not able to be determined here, despite the appearance of many dissociation products. This is likely due to the presence of additional post-translational modifications or proteolytic processing events that were not accounted for in the annotated database used here.

Note that identical protein identities were determined upon searching the entire protein database against the experimentally observed product ion masses only, i.e., without constraints placed on the parent mass. Also, the search tolerance of ±5 Da used for matching the experimentally determined product ion masses with the masses of predicted product ions for each of the proteins identified here was larger than the mass errors found in the experimentally derived data, which were generally within 2-3 Da of the predicted values. Thus, when using product ion data in conjunction with an intact protein mass, even with the relatively poor mass accuracy observed here, the correct proteins could be readily identified and distinguished from other proteins which differ in mass by less than 1 Da. It is important to recognize that in the current instrumentation, mass accuracy is limited by undersampling of the peaks when the mass range of the ion trap is extended beyond its usual upper mass limit of 650 Da, and not due to any inherent limitations introduced by the use of ion/ion reactions for precursor and product ion charge state manipulation. Of the four proteins identified here, CspC, hdeA and hdeB have all been previously observed on 2D gels as relatively abundant spots. Additionally, the removal of the signal peptides from hdeA and hdeB have been confirmed previously by Link et al. via N-terminal Edman degradation following separation of these proteins by 2D gel electrophoresis (Link et al., Electrophoresis. 1997, 18, 1259-1313). CspE and CspC have also been identified by peptide mass fingerprinting of their proteolytic digests following fractionation by RP-HPLC of a soluble protein extract of E. coli (Dai et al., Rapid Commun. Mass Spectrom. 1999, 13, 73-78).

TABLE 1
Summary of the database search results for proteins
from the soluble whole cell lysate HPLC fraction of E. coli.

| Mass [M + H]⁺: | 7331 | | | | | | | |
|---|---|---|---|---|---|---|---|---|
| Identity: | Cold shock-like protein cspE (CSPE_ECOLI) | | | | | | | |
| Sequence: | SKIKGNVKWFNESKGFGFITPEDGSKDVFVHFSAIQTNGFKTLAEGQRVEFEITNGAKSAANVIAL | | | | | | | |
| Matches: | 56 of 60 (14 of 60)^{a)} | | | | | | | |
| Score: | 249.58 (56.03)^{b)} | | | | | | | |
| Matched Product Ions: | 1649 b₁₄, | 1853 b₁₆, | 1910 b₁₇, | 2057 b₁₈, | 2171 b₁₉ | 2272 b₂₀, | 2498 b₂₂ | 2613 b₂₃, |
| | ^{c)}2757 b₂₅, | ^{c)}2885 b₂₆, | 3000 b₂₇, | 3346 b₃₀ | 3788 b₃₄, | 3901 b₃₅, | 4030 b₃₆, | 4131 b₃₇, |
| | 4245 b₃₈, | ^{c)}4449 b₄₀, | ^{c)}4577 b₄₁, | 4992 b₄₅, | 5561 b₅₀, | 5951 b₅₃, | 6052 b₅₄, | 6166 b₅₅, |
| | ^{c)}6479 b₅₉, | 6806 b₆₃, | 6920 b₆₄, | 7019 b₆₅, | 7132 b₆₆, | 7203 b₆₇, | 1773 y₁₈, | 2343 y₂₃, |
| | ^{c)}2757 y₂₇, | ^{c)}2885 y₂₈, | 3305 y₃₂ | 3704 y₃₆, | 3851 y₃₇ | 3988 y₃₈, | 4334 y₄₁, | ^{c)}4449 y₄₂, |
| | ^{c)}4577 y₄₃, | 4721 y₄₅, | 4836 y₄₆, | 5063 y₄₈, | 5164 y₄₉, | 5277 y₅₀, | 5424 y₅₁, | 5481 y₅₂, |
| | 5685 y₅₄, | 5814 y₅₅, | 5901 y₅₆, | 6030 y₅₇, | 6291 y₅₉, | ^{c)}6477 y₆₀, | 6605 y₆₁, | 6876 y₆₄ |
| Mass [M + H]⁺: | 7273 | | | | | | | |
| Identity: | Cold shock-like protein cspC (CSPC_ECOLI) | | | | | | | |
| Sequence: | AKIKGQVKWFNESKGFGFITPADGSKDVFVHFSAIQGNGFKTLAEGQNVEFEIQDGQKGPAAVNVTAI | | | | | | | |
| Matches: | 27 of 29 (8 of 29)^{a)} | | | | | | | |
| Score: | 137.25 (35.37)^{b)} | | | | | | | |
| Matched Product Ions | 3423 b₃₁ | 3970 b₃₆, | 4345 b₄₀ | 4687 b₄₃, | 4758 b₄₄, | 4888 b₄₅, | 5073 b₄₇, | 5187 b₄₈, |
| | 5286 b₄₉, | 5415 b₅₀, | 5691 b₅₂, | 5805 b₅₃, | 5933 b₅₄, | 6048 b₅₅, | ^{c)}6233 b₅₇, | 6361 b₅₈, |
| | ^{c)}6418 b₅₉, | 6586 b₆₁, | 6657 b₆₂, | 6757 b₆₃, | 6871 b₆₄, | 6970 b₆₅, | ^{c)}7071 b₆₆, | 4720 y₄₅, |
| | ^{c)}6232 y₅₉, | ^{c)}6418 y₆₀, | ^{c)}7072 y₆₆ | | | | | |
| Mass [M + H]⁺: | 9742 | | | | | | | |
| Identity: | Protein hdeA (HDEA_ECOLI) | | | | | | | |
| Sequence: | ADAQKAADNKKPVNSWTCEDFLAVDESFQPTAVGFAEALNNKDKPEDAVLDVQGIATVTPAIVQA | | | | | | | |
| | CTQDKQANFKDKVKGEWDKIKKDM | | | | | | | |
| Matches: | 14 of 13 (5 of 13)^{a)} | | | | | | | |
| Score: | 449.35 (63.27)^{b)} | | | | | | | |
| Matched Product Ions | 5508 b₅₁, | ^{c)}7306 b₆₉, | 8023 b₇₅, | 8138 b₇₆, | 8493 b₇₉, | 8680 b₈₁, | ^{c)}9350 b₈₆, | 9479, b₈₇, |
| | 9594 b₈₈, | ^{c)}7307 y₆₇, | 8600 y₇₈, | 8970 y₈₁, | ^{c)}9356 y₈₅, | 9484 y₈₆ | | |
| Mass [M + H]⁺: | 9065 | | | | | | | |
| Identity: | Protein hdeB (HDEB_ECOLI) | | | | | | | |
| Sequence: | ANESAKDMTCQEFIDLNPKAMTPVAWWMLHEETVYKGGDTVTLNETDLTQIPKVIEYCKKNPQK | | | | | | | |
| | NLYTFKNQASNDLPN | | | | | | | |
| Matches: | 9 of 11 (2 of 11)^{a)} | | | | | | | |
| Score: | 429.16 (5.35)^{b)} | | | | | | | |
| Matched Product Ions: | ^{c)}6861 b₆₀, | 8095 b₇₀, | 8409 b₇₃, | 8610 b₇₅, | 8725 b₇₆, | 8838 b₇₇, | 8935 b₇₈, | ^{c)}6858 y₅₉, |
| | 8350 y₇₂ | | | | | | | |

^{a)}The number of matches of the second ranked protein obtained from the modified SWISSPROT database search are indicated in parenthesis.
^{b)}The scores of the second ranked proteins obtained from the modified SWISSPROT database search are indicated in parenthesis.
^{c)}Product ion masses matching both b- and y-type ions, within the database search tolerance of ±5 Da, are indicated in italics.

The product ion spectra shown here are generally consistent with an emerging picture of the fragmentation behavior of multiply charged protein ions in the gas-phase. Several recent studies have shown that the fragmentation of whole protein ions under ion trap collisional activation conditions is strongly influenced by the precursor ion charge state as well as the total number of basic sites in the amino acid sequence (Cargile et al., Anal. Chem. 2001, 73, 1277-1285; Reid et al., Anal. Chem. 2001, 74, 577-583; Wells et al., Int. J. Mass Spectrom. 2000, 203, A1-A9; Reid et al., Anal. Chem. 2001, 73, 3274-3281; Wells et al., J. Am. Soc. Mass Spectrom. 2001, 12, 873-876; Engel et al., Int. J. Mass Spectrom. 2001, In Press; Chrisman et al., Rapid Commun. Mass Spectrom. 2001, 15, 2334-2340). Generally, intermediate charge states give rise to the most extensive non-specific cleavage of the protein backbone, often allowing derivation of a sequence tag for subsequent database searching (Cargile et al., Anal. Chem. 2001, 73, 1277-1285). At other charge states, the facile loss of NH₃ or H₂O (very low charge states corresponding to less than the number of arginine residues), preferential cleavage at the C-terminal of aspartic acid and lysine residues (low charge states), and preferential cleavage at the N-terminal of proline residues (high charge states), are often the dominant fragmentation products observed (Reid et al., Anal. Chem. 2001, 73, 3274-3281; Engel et al., Int. J. Mass Spectrom. 2001, In Press; Newton et al., Int. J. Mass Spectrom. 2001, 212, 359-376).

For example, the first of the proteins identified in this study, cold shock like protein E (CspE) (FIG. 5A and Table 1), has a total of 10 basic sites in its amino acid sequence, including one arginine residue, so the +5 charge state examined here falls into the intermediate range. In keeping with the trends described above, extensive non-specific fragmentation of this charge state was observed. Additionally, the abundant b₂₃/y₄₅ and b₂₀/y₄₈ complementary product ion pairs observed in the post-ion/ion MS/MS spectrum correspond to specific cleavages at the C-terminal of aspartic acid as well as the N-terminal of proline, respectively

Conclusions. The results described here indicate that the dissociation of whole protein ions can provide sufficient information to enable identification of the protein via database searching of the uninterpreted product ion spectra. We have developed an approach that takes advantage of gas-phase ion chemistry to reduce the reliance on condensed-phase chemistries and separations for the identification of proteins present in complex mixtures. The present approach relies heavily on gas-phase ion/ion proton transfer reactions for precursor and product ion charge state manipulation, and intact protein ion dissociation reactions coupled with database searching for protein identification. The ability to inhibit ion/ion proton transfer rates in a mass-to-charge dependant fashion, along with the ability to isolate ions on a mass-to-charge basis, enables multiply-charged protein ions present in a complex mixture to be concentrated and charge-state purified. This capability is illustrated here for five components of a mixture containing approximately thirty proteins derived from a whole cell lysate of E. coli.

The ability to concentrate and purify proteins ions in the gas-phase is particularly useful in “top down” protein identification/characterization strategies. Once a protein has been purified and concentrated into a single charge state, dissociation of the resulting precursor ion population can provide sufficient information to enable identification of the protein via database searching. Four of the five proteins subjected to concentration, purification and dissociation in this work could be identified by matching from a partially annotated E. coli protein sequence database. The fifth protein is apparently not present as a distinct entity in the current version of the database. It may be a fragment of a database entry, for example. Further refinements to the database search strategy may allow protein identification from less than fully annotated databases, including the ability to identify and characterize protein containing otherwise unknown post-translational modifications.

The approach used here to identify proteins by database matching of protein ion fragmentation data does not rely on a priori interpretation of the product ion spectrum. Such an approach facilitates possible automation of the protein identification process. The use of ion abundances in conjunction with weighting factors for fragmentations occurring at known preferential cleavage sites improves the discriminatory utility of the scoring algorithm. Further experience of the charge state dependant fragmentation behavior of protein ions will allow further refinement of the scoring algorithm.

Finally, the protein size of amenable to study and the specificity with which proteins can be identified and characterized were limited by the mass analysis performance characteristics of the ion trap used in this work. The overall concentration/purification/dissociation process and the data-base search approach described herein, however, are not constrained to any particular form of mass analysis. Improved mass analysis characteristics, obtained either with a higher performance ion trap or some other form of mass analysis, can directly translate to several performance improvements in the analysis of complex protein mixtures, including specificity and protein size amenable to study.

The complete disclosures of all patents, patent applications including provisional patent applications, and publications, and electronically available material (e.g., GenBank amino acid and nucleotide sequence submissions) cited herein are incorporated by reference. The foregoing detailed description and examples have been provided for clarity of understanding only. No unnecessary limitations are to be understood therefrom. The invention is not limited to the exact details shown and described; many variations will be apparent to one skilled in the art and are intended to be included within the invention defined by the claims.

1. A method for identifying a protein of interest comprising:
subjecting the protein of interest to tandem mass spectrometry to cause the protein of interest to fragment into a multiplicity of product ions having experimentally determined product ion masses;
comparing the experimentally determined product ion masses with product ion masses calculated for each member of a comparison set comprising database protein sequences, so as to identify for each member of the comparison set the product ion matches within a predetermined mass tolerance; and
discriminating between possible matches to members of the comparison set on the basis of experimentally observed product ion abundances to identify the protein of interest.

2. The method of claim 1 wherein the product ion masses calculated for the members of the comparison set include product ion masses calculated for database protein sequences that are modified to account for at least one known or predicted protein structural modification.

3. The method of claim 1 or 2 wherein the protein of interest is present in a mixture comprising a multiplicity of proteins, and wherein subjecting the protein of interest to tandem mass spectrometry comprises subjecting the mixture of proteins to tandem mass spectrometry.

4. The method of claim 1 or 2 wherein the comparison set comprises some or all of the protein sequences or subsequences included in the protein database.

5. The method of claim 1 or 2 wherein the comparison set comprises protein sequences having a calculated mass that matches the mass of the protein of interest within a predetermined mass tolerance.

6. The method of claim 1 or 2 wherein the comparison set comprises protein subsequences having a calculated mass that matches the mass of the protein of interest within a predetermined mass tolerance.

7. The method of claim 1 or 2 wherein the comparing step comprises:
determining mass differences between selected pairs of experimentally determined product ion masses;
determining mass differences between selected pairs of calculated product ion masses; and
comparing the mass differences between the selected pairs of experimentally determined product ion masses with the mass differences between selected pairs of calculated product ion masses so as to identify for each member of the comparison set the product ion matches within a predetermined mass tolerance.

8. The method of claim 7 wherein the protein of interest is not accurately reflected in the protein database.

9. The method of claim 1 or 2 further comprising calculating, for each member of the comparison set, a score comprising a weighted sum of the product ion mass matches based on experimentally observed product ion abundances.

10. The method of claim 7 further comprising calculating, for each member of the comparison set, a score comprising a weighted sum of the product ion mass matches based on experimentally observed product ion abundances.

11. The method of claim 7 wherein the pairs of product ions selected for the determination of mass differences are selected on the basis of favored cleavage sites.

12. The method of claim 1 or 2 further comprising calculating a score for each member of the comparison set, wherein the score comprises a weighted sum of the product ion mass matches based on favored cleavage sites.

13. The method of claim 12 wherein weighting factors assigned to the favored cleavage sites vary with the identity of the amide bond.

14. The method of claim 12 wherein the protein of interest is ionized to yield a multiply charged protein precursor ion prior to fragmentation, and wherein the weighting factors assigned to the favored cleavage sites vary with the charge state of the protein precursor ion.

15. The method of claim 12 wherein the protein of interest is ionized to yield a multiply charged protein precursor ion prior to fragmentation, and wherein the weighting factors assigned to the favored cleavage sites vary with the charge polarity of the protein precursor ion.

16. The method of claim 1 or 2 wherein the protein of interest comprises a disulfide linkage, and wherein the multiplicity of product ions comprises at least one z-S ion.

17. A method for identifying a protein of interest comprising:
subjecting the protein of interest to tandem mass spectrometry to cause the protein of interest to fragment into a multiplicity of product ions having experimentally determined product ion masses;
determining mass differences between selected pairs of experimentally determined product ion masses;
determining mass differences between selected pairs of product ion masses calculated for each member of a comparison set comprising database protein sequences; and
comparing the mass differences between the selected pairs of experimentally determined product ion masses with the mass differences between selected pairs of calculated product ion masses so as to identify for each member of the comparison set the product ion matches within a predetermined mass tolerance, to identify the protein of interest.

18. The method of claim 17 wherein the product ion masses calculated for the members of the comparison set include product ion masses calculated for database protein sequences that are modified to account for at least one known or predicted protein structural modification.

19. The method of claim 17 wherein the comparison set comprises some or all of the protein sequences or subsequences included in the protein database.

20. The method of claim 17 wherein the protein of interest is not accurately reflected in the protein database.

21. The method of claim 17 further comprising discriminating between possible matches to members of the comparison set on the basis of experimentally observed product ion abundances to identify the protein of interest.

22. The method of claim 21 further comprising calculating a score for each member of the comparison set, wherein the score comprises a weighted sum of the product ion mass matches based the experimentally observed product ion abundances.

23. The method of claim 17 wherein the pairs of product ions selected for the determination of mass differences are selected on the basis of favored cleavage sites.

24. The method of claim 23 further comprising calculating a score for each member of the comparison set, wherein the score comprises a weighted sum of the product ion mass matches based on favored cleavage sites.

25. The method of claim 24 wherein weighting factors assigned to the favored cleavage sites vary with the identity of the amide bond.

26. The method of claim 24 wherein the protein of interest is ionized to yield a multiply charged protein precursor ion prior to fragmentation, and wherein the weighting factors assigned to the favored cleavage sites vary with the charge state of the protein precursor ion.

27. The method of claim 24 wherein the protein of interest is ionized to yield a multiply charged protein precursor ion prior to fragmentation, and wherein the weighting factors assigned to the favored cleavage sites vary with the charge polarity of the protein precursor ion.

28. A method for identifying a protein of interest comprising a disulfide bond, the method comprising:
subjecting the protein of interest to tandem mass spectrometry to cause the protein of interest to fragment into a multiplicity of product ions having experimentally determined product ion masses, said multiplicity of product ions comprising at least one z-S product ion;
comparing the experimentally determined product ion masses with product ion masses calculated for each member of a comparison set comprising database protein sequences, so as to identify for each member of the comparison set the product ion matches within a predetermined mass tolerance, to identify the protein of interest.

29. The method of claim 28 further comprising calculating a score for each member of the comparison set, wherein the score comprises a weighted sum of the product ion mass matches based the experimentally observed product ion abundances.

30. The method of claim 28 further comprising calculating a score for each member of the comparison set, wherein the score comprises a weighted sum of the product ion mass matches based on product ion type.

31. A method for identifying a protein of interest comprising:
subjecting the protein of interest to tandem mass spectrometry to cause the protein of interest to fragment into a multiplicity of product ions having experimentally determined product ion masses, wherein the protein of interest is ionized to yield a multiply charged protein precursor ion prior to fragmentation;
comparing the experimentally determined product ion masses with product ion masses calculated for each member of a comparison set comprising database protein sequences, so as to identify for each member of the comparison set the product ion matches within a predetermined mass tolerance; and
calculating a score for each member of the comparison set, wherein the score comprises a weighted sum of the product ion mass matches based on favored cleavage sites, and wherein the weighting assigned to said favored cleavage sites depends upon the charge state of the protein precursor ion.
